(19) United States
(12) Patent Application Publication
(10) Pub. No.: US 2006/0013790 A1
(43) Pub. Date: Jan. 19, 2006

(54) Cosmetic composition comprising a defined silicone polymer and a gelling agent

(75) Inventor: Momoko Shimizu, Kawasaki-shi (JP)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/181,950

(22) Filed: Jul. 15, 2005

Related U.S. Application Data
(60) Provisional application No. 60/591,930, filed on Jul. 29, 2004.

(30) Foreign Application Priority Data
Jul. 16, 2004 (FR) 04 51557

(51) Int. Cl. A61K 8/894

(52) U.S. Cl. 424/70.12

(57) ABSTRACT

The present invention relates primarily to a cosmetic makeup and/or care composition intended for application to the skin, lips and/or epidermal derivatives, comprising a particular silicone polymer, a liquid fatty phase and a gelling agent for said liquid fatty phase.

The present invention relates to a cosmetic makeup and/or care composition intended for application to the skin, lips and/or epidermal derivatives, comprising a particular silicone polymer, a liquid fatty phase and a gelling agent for said liquid fatty phase.

The composition exhibits in particular an improved colour permanence, as well as a satisfactory or even improved gloss and a satisfactory or even improved comfort.

The cosmetic compositions to which the present invention is directed are, more particularly, makeup and/or care products intended for application to the skin, lips and/or epidermal derivatives, especially lipsticks, lip balms, lip pencils, liquid foundations or solid foundations, cast in particular as a stick or in a dish, concealer products and skin colouring products, temporary tattoos, eye makeup products such as eyeliners, in particular in the form of pencils, and mascaras, in particular in the form of cakes, or else eyeshadows.

Compositions comprising silicone alkylglyceryl ether derivatives have been described in applications JP 6-305933 and JP 7-330547. Compositions having an oily base and comprising polyglycerylated silicone derivatives or fluoroalkylpolyglycerylated silicone derivatives have been proposed in applications JP 6-157236, JP 9-071504 and JP 10-310504. Compositions comprising silicone alkylglycerol derivatives have also been described in patent EP 0 475 130 and in applications JP 2-844453 and JP 2-587797. Other compositions comprising silicone derivatives hydroxylated with saccharides, with butylene glycol or with glycerol have been described in applications JP 5-186596 and JP 6-145023.

For the purposes of the present invention the term “keratin materials” is intended to embrace the skin, mucosae, such as the lips, the nails and the keratin fibres, as exemplified by the eyelashes and the hair.

The cosmetic compositions in accordance with the present invention are particularly advantageous for use on the skin and lips.

The inventors have thus found, unexpectedly, that the use of a silicone polymer of general formula (I):
R¹ₐR²_{b}R³_{c}SiO_{(4-a-b-c)/2} (I)
in which R¹ is in particular an alkyl radical, R² is represented by the general formula (III):
Q-O—X (III)
in which Q is a divalent hydrocarbon radical and X is a polyhydroxylated hydrocarbon radical,
and R³ is an organosiloxane group of general formula (IV): 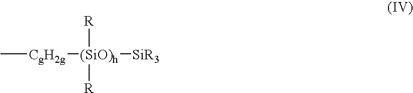
in which the radicals R each represent, independently of one another, an alkyl radical, in combination with at least one fatty-phase gelling agent is particularly advantageous for the formulation of cosmetic compositions exhibiting an improved staying power on keratin materials, without detriment to the gloss, or even with a likewise-improved average gloss, and possibly liable to exhibit improved comfort.

Accordingly, according to one of its first aspects, the invention provides an anhydrous cosmetic composition comprising at least one liquid fatty phase, at least one non-crystalline, non-silicone gelling agent for said liquid fatty phase, and at least one silicone polymer of general formula (I):
R¹ₐR²_{b}R³_{c}SiO_{(4-a-b-c)/2} (I)
in which:
a, b and c are such that a varies from 1 to 2.5 and b and c independently of one another vary from 0.001 to 1.5,
R¹, which is identical or different at each occurrence, is selected from:
   C₁ to C₃₀ alkyl radicals, substituted where appropriate by one or more fluorine atoms and/or amino and/or carboxyl groups,
   aryl and aralkyl radicals,
   radicals of general formula (II):
      —C_{d}H_{2d}—O—(C₂H₄O)ₑ(C₃H₆O)_{f}R⁴ (II)
      with:
      R⁴ being a C₁ to C₃₀ hydrocarbon radical or a radical R⁵—(CO)—, with R⁵ being a C₁ to C₃₀ hydrocarbon radical, and
      d, e and f being integers such that d varies from 0 to 15 and e and f, independently of one another, vary from 0 to 50, and
   combinations thereof,
R² is a radical represented by the general formula (III):
   -Q-O—X (III)
   with:
   Q being a divalent C₂ to C₂₀ hydrocarbon radical which can include at least one ether bond and/or at least one ester bond, and
   X being a polyhydroxylated hydrocarbon radical,
R³ is an organosiloxane of general formula (IV): 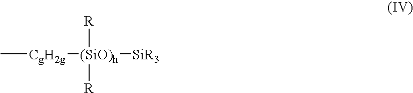
   with:
   the radicals R each representing, independently of one another, a radical selected from C₁ to C₃₀ alkyl radicals, substituted where appropriate by one or more fluorine atoms, and aryl and aralkyl radicals,
   g and h being integers such that g varies from 1 to 5 and h varies from 0 to 500.

According to a second of its aspects the present invention provides a cosmetic composition comprising at least one colorant, at least one inorganic gelling agent and at least one silicone polymer of general formula (I), as defined above.

According to a third of its aspects the present invention also provides an anhydrous cosmetic composition comprising at least one liquid fatty phase, said liquid fatty phase containing at least one silicone oil, at least one gelling agent for said liquid fatty phase, and at least one silicone polymer of general formula (I), as defined above.

According to a fourth of its aspects the present invention further provides a cosmetic composition comprising at least one liquid fatty phase, at least one liquid-fatty-phase gelling agent, at least one surfactant and at least one silicone polymer of general formula (I), as defined above.

According to a fifth of its aspects the present invention additionally provides an anhydrous cosmetic composition comprising at least one liquid fatty phase, at least one gelling agent for said liquid fatty phase, and at least one silicone polymer of general formula (I), as defined above, with the proviso that, when R² is a group of general formula (IIIA):
—C₃H₆—O[CH₂CH(OH)CH₂O]ₙH (IIIA)
in which n is an integer varying from 1 to 5, R¹ is not a C₁₂ alkyl radical.

According, further, to a sixth of its aspects the present invention provides a cosmetic composition as defined above which is intended for making up and/or caring for the lips and/or the skin, and in particular provides a lipstick.

According to yet another of its aspects the present invention also provides a method of making up and/or caring for keratin materials, and particularly the skin and/or lips, which comprises applying thereto at least one cosmetic composition in accordance with the present invention.

According to another of its aspects the invention provides a synthetic support on which is present, over some or all of its surface, at least one layer of a composition according to the invention.

According to yet another of its aspects the invention provides for the use of at least one silicone polymer of general formula (I), as defined above, in combination with at least one liquid-fatty-phase gelling agent for preparing a cosmetic composition exhibiting improved staying power, in combination with an average gloss which is satisfactory or even improved.

By improved staying power is meant improved water resistance and/or oil resistance and/or reduced transfer.

The cosmetic compositions according to the invention may be in the form of a paste, liquid, gel, cream or solid. In particular the cosmetic compositions according to the invention are in cast form, and more particularly they are in the form of a stick. They may also be in the form of a simple oil-in-water or water-in-oil emulsion or multiple emulsion, or of an anhydrous, solid or flexible gel.

In particular they are in an anhydrous form.

The term “composition in cast form” denotes for the purposes of the present invention a solid or semi-solid composition obtained after cooling a composition introduced in the melt state into a mould. The compositions may be cast in the form of a stick or crayon, or in a dish.

According to one particular embodiment the cosmetic composition according to the invention is in cast form, i.e., in solid or semi-solid form, and more particularly in the form of a stick.

In order to determine the hardness of a cosmetic composition in accordance with the invention a stick of said composition is prepared which has a circular section 12.7 mm in diameter. The stick is cast and then kept at a temperature of 20° C., 24 hours before measurement.

The hardness can be measured by the “cheese wire” method, which consists in cutting the stick transversely by means of a rigid tungsten wire 250 μm in diameter, by advancing the wire relative to the stick at a speed of 100 mm/min. The hardness corresponds to the maximum shearing force exerted by the wire on the stick at 20° C., this force being measured by means of a DFGS2 dynamometer sold by Indelco-Chatillon. The hardness is expressed in grams.

According to this method the hardness of a cosmetic composition in accordance with the invention in the form of a stick varies in particular from 50 to 300 g, especially from 70 to 250 g and more particularly from 100 to 230 g.

One of the advantages of the cosmetic compositions according to the present invention is that of exhibiting improved colour permanence, as manifested in particular by reduced migration or transfer of colour, and/or improved water resistance of the colour and/or improved oil resistance of the colour, and/or reduced migration during application of the makeup.

Another advantage of the cosmetic compositions according to the present invention is the maintenance of a comfortable sensation and of an absence of a sticky sensation while exhibiting effective adhesion to the skin.

A third advantage of the cosmetic compositions according to the present invention is the maintenance of the aesthetic effect, particularly the gloss effect, over time.

According to a fourth advantage the cosmetic compositions according to the invention make it possible to impart a soft and smooth sensation and to maintain effective moisturizing.

According to a fifth advantage the cosmetic compositions according to the present invention exhibit good staying power with regard to external factors liable to modify their aesthetic properties, such as perspiration or a meal in the case of a lipstick.

Silicone Polymer of General Formula (1)

The silicone polymers in accordance with the silicone polymer of general formula (I) and which can be used in the cosmetic compositions according to the present invention are described in detail in patent application EP 1 213 316, which is incorporated by reference in the present application.

The silicone polymers of general formula (I) have the advantage of being useful as a surfactant and/or as an oily base.

Introduced in sufficient quantity, they have the advantage of imparting an improvement in staying power or even in gloss and/or in comfort to the cosmetic compositions according to the invention.

In particular the silicone polymers which can be used in the cosmetic compositions according to the present invention are represented by the general formula (I) below:
R¹ₐR²_{b}R³_{c}SiO_{(4-a-b-c)/2} (I)
in which:
a) a, b and c are such that a varies from 1 to 2.5; and b and c independently of one another vary from 0.001 to 1.5.
b) R¹, which is identical or different at each occurrence, is selected from:
   C₁ to C₃₀ alkyl radicals, substituted where appropriate by one or more fluorine atoms and/or amino and/or carboxyl groups,
   aryl and aralkyl radicals,
   radicals of general formula (II):
      —C_{d}H_{2d}—O—(C₂H₄O)ₑ(C₃H₆O)_{f}R⁴ (II)
      in which:
      R⁴ is a C₁ to C₃₀ hydrocarbon radical or a radical R⁵—(CO)—, with R⁵ being a C₁ to C₃₀ hydrocarbon radical, and
      d, e and f are integers such that d varies from 0 to 15 and e and f, independently of one another, vary from 0 to 50, and
   combinations thereof.
c) R² is represented by the general formula (III) below:
   -Q-O—X (III)
   in which:
   Q is a divalent C₂ to C₂₀ hydrocarbon radical which can include at least one ether bond and/or at least one ester bond, and
   X is a polyhydroxylated hydrocarbon radical.
d) R³ is an organosiloxane group of general formula (IV): 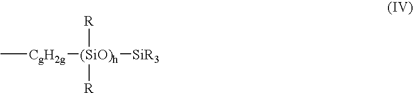
   with:
the radicals R each representing, independently of one another, a radical selected from C₁ to C₃₀ alkyl radicals, substituted where appropriate by one or more fluorine atoms, and aryl and aralkyl radicals,
g and h being integers such that g varies from 1 to 5 and h varies from 0 to 500.

When the radicals R represent a radical selected from C₁ to C₃₀ alkyl radicals substituted where appropriate by one or more fluorine atoms, and from aryl radicals and aralkyl radicals, they have the same meaning as the radical R¹ as defined above.

It should be noted that the radicals R¹, R² and R³ of the silicone polymers of general formula (I), as defined above, are distributed randomly or statistically; that is, they appear in the structure of the polymer without a determined order. Similarly, R¹, R², and R³ may respectively feature radicals of different kind in a compound of general formula (I).

In one particular embodiment,

in a):
more particularly a varies from 1.2 to 2.3. And, in particular, b and c, independently of one another, vary from 0.05 to 1.

in b):
when R¹ is an alkyl radical, it may be a C₁ to C₃₀ alkyl radical, in particular a C₁ to C₂₅ alkyl radical, more particularly a C₁ to C₂₀ alkyl radical, in particular a C₁ to C₁₀ alkyl radical, and especially a C₁ to C₆ alkyl radical, and in particular a C₁ to C₄ alkyl radical. More particularly it may be a methyl, ethyl, n- or isopropyl, n- or iso- or tert-butyl, pentyl, hexyl, heptyl, octyl, nonyl, decyl, undecyl or lauryl radical. It may also be a cycloalkyl radical such as a cyclopropyl, a cyclobutyl, a cyclopentyl or a cyclohexyl. It may also be a linear or branched, monounsaturated or polyunsaturated alkyl radical. It may also be an alkyl radical substituted by one or more fluorine atoms, such as trifluoropropyl or heptadecafluorodecyl. It may also be an alkyl radical substituted by one or more amino groups, such as 2-aminoethyl, 3-aminopropyl, and (2-ethylamino)-3-aminopropyl. It may also be an alkyl group substituted by one or more carboxyl groups, such as 3-carboxypropyl.
R¹ may also be an aryl or aralkyl radical such as the phenyl radical, the tolyl radical, the benzyl radical and the phenethyl radical.
R¹ may also be an organic group represented by the general formula (II):
   —C_{d}H_{2d}—O—(C₂H₄O)ₑ(C₃H₆O)_{f}R⁴ (II)

According to one particular embodiment R¹ may be a hydroxylated radical or a radical obtained from the addition reaction of a saturated or unsaturated, linear or branched alkenyl ether in which d=0 and which is therefore of formula:
—O—(C₂H₄O)ₑ(C₃H₆O)_{f}R₄

In this case, when e and f are zero, R¹ is an alkoxy group having from 4 to 30 carbon atoms, for example a C₄ to C₁₀ lower alkoxy radical, such as butoxy or pentoxy, or a C₁₁ to C₃₀ higher alkoxy radical, such as oleoxy, stearoxy, viz., for example, cetyl alcohol, oleyl alcohol and stearyl alcohol, or a radical obtained from an acid or a fatty acid, such as acetic acid, lactic acid, butyric acid, oleic acid, stearic acid and behenic acid.

When e and f are greater than 1, R¹ is a hydroxyl radical originating from the addition reaction of an alkylene oxide.

When e and f are zero it is particularly advantageous for d to be 3, 5 or 11. In this case R¹, depending on the nature of the substituent R⁴, is an allyl ether, pentenyl ether or undecenyl ether radical or an allyl stearyl ether, pentenyl behenyl ether or undecenyl oleyl ether radical.

When e or f are other than zero, an alkoxy radical and an ester radical are present via a polyoxyalkylene group.

Irrespective of what e and f are, it is particularly advantageous for d to be within the range varying from 3 to 5.

According to one embodiment the radical R¹ may be any one of the above-defined radicals or a combination of two or more of these radicals.

Advantageously R¹ is an alkyl radical selected from the methyl radical, the lauryl radical, and combinations thereof.

Moreover, when R¹ represents two or more radicals in a single general formula (I), a methyl radical and a lauryl radical, for example, these radicals appear in the structure at random and with a frequency which is specific to them.

In particular, at least 50% of the radicals R¹, especially at least 70% of the radicals R¹, and more particularly 100% of the radicals R¹ are methyl radicals.

In c):
Q may in particular be a divalent hydrocarbon radical selected from:
—(CH₂)₂—, —(CH₂)₃—, —CH₂CH(CH₃)—CH₂, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₂—CH(CH₂CH₂CH₃)—, —CH₂—CH(CH₂CH₃)—, —(CH₂)₃—O—(CH₂)₂—, —(CH₂)₃—O—(CH₂)₂—O—(CH₂)₂—, —(CH₂)₃—O—CH₂CH(CH₃)— and —CH₂—CH(CH₃)—COO(CH₂)₂—.

Advantageously Q is a divalent radical selected from —(CH₂)₂— and CH₂)₃—.
X may particularly be a polyhydroxylated hydrocarbon radical containing at least two hydroxyl residues, and in particular a hydrocarbon group selected from glyceryl derivatives and saccharide derivatives.

The glycerol residues may be compounds having the following formulae, in which Q has the same meaning as in the general formula (III), and s and t are integers from within the range varying from 1 to 20, in particular from 1 to 15, in particular from 1 to 10, and more particularly from 1 to 5.

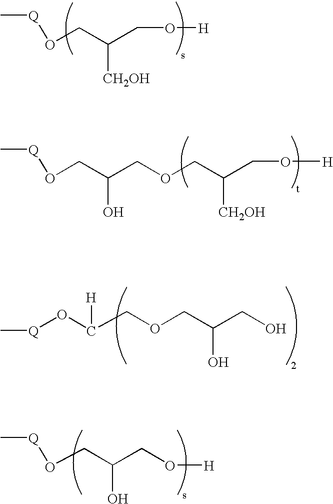

In the above formulae, one or more hydroxyl groups may be replaced by alkoxy groups or ester groups.

The saccharide radicals which can be used in the general formula (III) may be of monosaccharide type, such as glycosyl, mannosyl, galactosyl, ribosyl, arabinosyl, xylosyl or fructosyl groups, of oligosaccharide type, such as maltosyl, cellobiosyl, lactosyl or maltotriosyl, or of polysaccharide type, such as cellulose or starch.

In particular the saccharide groups are of monosaccharide or oligosaccharide type.

In d):
each of the radicals R may represent in particular, independently of one another, a radical selected from C₁ to C₂₀, more particularly C₁ to C₁₀, in particular C₁ to C₆ alkyl radicals, substituted where appropriate by one or more fluorine atoms. When the radicals R represent a radical selected from alkyl radicals as defined above, substituted where appropriate by one or more fluorine atoms, they have the same meaning as the radical R¹ as defined above.
g, according to one particular embodiment, is 2
h, according to one particular embodiment, is within the range varying from 1 to 50.

However, according to certain of the specific aspects of the present invention, as defined above, and in particular according to a fifth aspect, the silicone polymer of general formula (I) is such that, when the radical R² is represented by the general formula (IIIA):
—C₃H₆O[CH₂CH(OH)CH₂O]ₙH (IIIA)
in which n varies from 1 to 5, the radical R¹ is other than a C₁₂ alkyl radical.

According to one particular embodiment the silicone polymer of general formula (I) that is suitable for the implementation of the present invention is such that:
a varies from 1 to 1.4, and b and c, independently of one another, vary from 0.02 to 0.03, and
R¹ is a C₁, to C₁₀, in particular C₁ to C₆, and more particularly C₁ to C₄ alkyl radical.
R² is represented by the formula (IIIA):
   C₃H₆O[CH₂CH(OH)CH₂O]ₙH (IIIA)
   in which:
n varies from 1 to 5, and
R³ is represented by the formula (IVA):
   —C₂H₄(CH₃)₂SiO[(CH₃)₂SiO]ₘ Si(CH₃)₃ (IVA)
   in which:
m varies from 3 to 9.

According to another particular embodiment the silicone polymer of general formula (I), which can be used in the cosmetic compositions according to the present invention, is such that:
a varies from 1 to 1.4 and b and c, independently of one another, vary from 0.02 to 0.04,
R¹ is a methyl radical,
R² is represented by the formula (IIIA) in which n varies from 1 to 5, and
R³ is represented by the formula (IVA) in which m varies from 3 to 9.

Advantageously the silicone polymer of general formula (I) used in the cosmetic composition in accordance with the invention may be selected from polyglyceryl-3 polymethylsiloxyethyl dimethicone, laurylpolyglyceryl-3 polymethylsiloxyethyl dimethicone and polyglyceryl-3 disiloxane dimethicone, whose formulae are, respectively:
polyglyceryl-3 polymethylsiloxyethyl dimethicone (formula (V)): 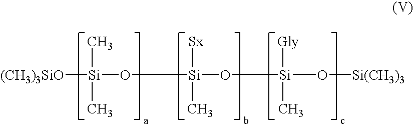
in which:
Sx: —C₂H₄ [(CH₃)₂SiO]ₘSi(CH₃)₃
Gly: —C₃H₆O[CH₂—CH(OH)CH₂O]ₙH
   with a=1-1.4, b=0.02-0.04, c=0.02-0.04, m=3-9, n=1-5,
   lauryl polyglyceryl-3 polymethylsiloxyethyl dimethicone (formula (VI)): 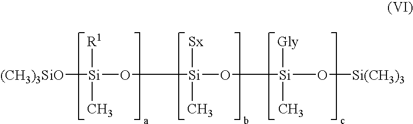
   in which Sx, Gly, a, b, c, m and n have the same meaning as above and R¹ is either a methyl radical or a lauryl radical.
   Polyglyceryl-3 disiloxane dimethicone (formula (VII)): 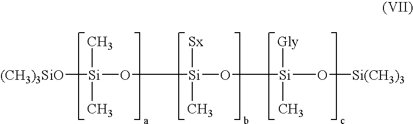
   in which Gly, a, b, c, m and n have the same meaning as above, and
   Sx: —O(CH₃)₂SiO—Si(CH₃)₃

The silicone polymer of general formula (I) may be present in the cosmetic compositions in accordance with the present invention in a proportion of from 0.1% to 40% by weight, in particular from 0.5% to 30% by weight, more particularly from 1% to 25% by weight, in particular from 5% to 20% by weight, in particular from 7% to 15% by weight, relative to the total weight of the composition.

According to one particular embodiment the silicone polymer of general formula (I) is advantageously selected from polymers sold by Shin-Etsu under the references KF6100®, KF6104® and KF6105®.

According to yet another embodiment the polymer sold under the reference KF6104® is particularly suitable for the preparation of the cosmetic compositions in accordance with the invention.

According to yet another embodiment the compound denoted under the reference KF6104®, sold by Shin-Etsu, is particularly suitable for preparing cosmetic compositions in accordance with the invention which exhibit improved staying power on keratin materials without detriment to the gloss or even with a likewise improved average gloss.

Surfactants

According to one particular embodiment the cosmetic compositions in accordance with the present invention further comprise at least one surfactant.

By surfactant is meant a compound possessed of at least one hydrophilic moiety and at least one hydrophobic moiety. Hydrophilic groups and hydrophobic groups are well known to the skilled person.

According to another embodiment the cosmetic compositions in accordance with the present invention may also further comprise at least one silicone surfactant different from the silicone polymer of general formula (I).

The surfactants useful in the compositions in accordance with the invention may be selected from hydrocarbon surfactants and silicone surfactants.

The additional surfactants may advantageously be selected from nonionic, anionic, cationic and amphoteric surfactants or else surfactant emulsifiers.

Reference may be made to “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of that reference, for anionic, amphoteric and nonionic surfactants.

Nonionic Surfactant

As a non-limitative representation of the nonionic surfactants which can be used additionally to the silicone polymer of general formula (I), alone or in a mixture, in the compositions according to the invention mention may be made in particular of the following:
oxyethylene and/or oxypropylene ethers (which can contain from 1 to 150 oxyethylene and/or oxypropylene groups) of glycerol;
oxyethylene and/or oxypropylene ethers (which can contain from 1 to 150 oxyethylene and/or oxypropylene group) of fatty alcohols (particularly of C₈-C₂₄, and preferably C₁₂-C₁₈, alcohol) such as the oxyethylene ether of cetearyl alcohol containing 30 oxyethylene groups (CTFA name “Ceteareth-30”) and the oxyethylene ether of the mixture of C₁₂-C₁₅ fatty alcohols, containing 7 oxyethylene groups (CTFA name “C₁₂₋₁₅ Pareth-7”, sold under the name “Neodol 25-7”® by Shell Chemicals);
fatty acid esters (particularly of C₈-C₂₄, and preferably C₁₆-C₂₂, acid) of polyethylene glycol (which can contain from 1 to 150 ethylene glycol units) such as PEG-50 stearate and PEG-40 stearate, sold under the name MYRJ 52P by ICI Uniqema;
fatty acid esters (particularly of C₈-C₂₄, and preferably of C₁₆-C₂₂, acid) of glycerol ethers which are oxyethylenated and/or oxypropylenated (and can contain from 1 to 150 oxyethylene and/or oxypropylene groups), such as the PEG-200 glyceryl monostearate sold under the name “Simulsol 220 TM” by SEPPIC; polyethoxylated glyceryl stearate containing 30 ethylene oxide groups, such as the product Tagat S sold by Goldschmidt, polyethoxylated glyceryl oleate containing 30 ethylene oxide groups, such as the product Tagat O sold by Goldschmidt, polyethoxylated glyceryl cocoate containing 30 ethylene oxide groups such as the product Varionic LI 13 sold by Sherex, polyethoxylated glyceryl isostearate containing 30 ethylene oxide groups, such as the product Tagat L sold by Goldschmidt, and polyethoxylated glyceryl laurate containing 30 ethylene oxide groups, such as the product Tagat I from Goldschmidt;
fatty acid esters (particularly of C₈-C₂₄, and preferably C₁₆-C₂₂, acid) and sorbitol ethers which are oxyethylenated and/or oxypropylenated (and can contain from 1 to 150 oxyethylene and/or oxypropylene groups), such as the polysorbate 60 sold under the name “TWEEN 60” by Uniqema;
copolymers of ethylene oxide and propylene oxide, the condensates of ethylene oxide and propylene oxide with fatty alcohols, amines or fatty amines which are polyethoxylated, having preferably 2 to 30 mol of ethylene oxide, polyglycerolated fatty amides containing on average 1 to 5 glycerol groups, polyglycerolated diglycol amides, fatty acid esters of sorbitan, optionally oxyethylenated, fatty acid esters of sucrose, polyoxyalkylenated fatty acid esters, optionally oxyalkylenated alkylpolyglycosides, alkylglucoside esters, N-alkylglucamine derivatives and N-acylmethylglucamine derivatives, and amine oxides;
dimethicone copolyol, such as that sold under the name “Q2-5220” by Dow Corning;
dimethicone copolyol benzoate (Finsolv SLB 101 and 201 from Fintex),
copolymers of propylene oxide and ethylene oxide, also called EO/PO polycondensates;
esters and ethers of saccharides, such as sucrose stearate, sucrose cocoate, sorbitan stearate and mixtures thereof such as Arlatone 2121 sold by ICI;
fatty acid esters (particularly of C₈-C₂₄, preferably C₁₆-C₂₂, acid) of polyol, particularly of glycerol or of sorbitol, such as glyceryl stearate, glyceryl stearate such as the product sold under the name Tegin M by Goldschmidt, glyceryl laurate such as the product sold under the name Imwitor 312 by Huls, polyglyceryl-2 stearate, sorbitan tristearate and glyceryl ricinoleate;
the cyclomethicone/dimethicone copolyol mixture sold under the name “Q2-3225C” by Dow Corning;
and mixtures thereof.

The EO/PO polycondensates are, more particularly, copolymers consisting of polyethylene glycol blocks and polypropylene glycol blocks, such as, for example, triblock polyethylene glycol/polypropylene glycol/polyethylene glycol polycondensates.

As EO/PO polycondensate which can be used according to the invention, mention may be made of the triblock polyethylene glycol/polypropylene glycol/polyethylene glycol polycondensates sold under the “Synperonic” names, such as “Synperonic PE/L44” and “Synperonic PE/F127”, by ICI.

Among the surfactants which can be used in the cosmetic compositions in accordance with the present invention, mention may be made of the emulsifying silicone elastomers or resins sold by Shin-Etsu, under references KSG-310, KSG-320, KSG-330 and KSG-340.

The term “emulsifying silicone elastomer” denotes a silicone elastomer comprising at least one hydrophilic chain other than a polyglycerolated chain.

In particular the additional emulsifying silicone elastomer may be selected from polyoxyalkylenated silicone elastomers.

The polyoxyalkylenated silicone elastomer is a crosslinked organopolysiloxane obtainable by a crosslinking addition reaction of diorganopolysiloxane containing at least one hydrogen bonded to the silicon and a polyoxyalkylene having at least two ethylenically unsaturated groups.

Advantageously the polyoxyalkylenated crosslinked organopolysiloxane is obtained by a crosslinking addition reaction (A1) of diorganopolysiloxane containing at least two hydrogens each bonded to a silicon, and (B1) of polyoxyalkylene having at least two ethylenically unsaturated groups, in the presence in particular (C1) of a platinum catalyst, as described, for example, in patents U.S. Pat. No. 5,236,986 and U.S. Pat. No. 5,412,004.

In particular the organopolysiloxane may be obtained by reacting polyoxyalkylene (especially polyoxyethylene and/or polyoxypropylene) containing dimethylvinylsiloxy end groups and methylhydropolysiloxane containing trimethylsiloxy end groups, in the presence of a platinum catalyst.

The organic groups bonded to the silicon atoms of the compound (A1) may be alkyl groups having 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl and 3,3,3-trifluoropropyl; aryl groups such as phenyl, tolyl and xylyl; substituted aryl groups such as phenylethyl; and substituted monovalent hydrocarbon groups such as an epoxy group, a carboxylate ester group or a mercapto group.

Compound (A1) may therefore be selected from methylhydropolysiloxanes containing trimethylsiloxy end groups, dimethylsiloxane-methylhydrosiloxane copolymers containing trimethylsiloxy end groups, cyclic dimethylsiloxane-methylhydrosiloxane copolymers, and dimethylsiloxane-methylhydrosiloxane-laurylmethylsiloxane copolymers containing trimethylsiloxy end groups.

Compound (C1) is the catalyst of the crosslinking reaction, and is, in particular, chloroplatinic acid, chloroplatinic acid-olefin complexes, chloroplatinic acid-alkenylsiloxane complexes, chloroplatinic acid-diketone complexes, platinum black, and platinum on a support.

Advantageously the polyoxyalkylenated silicone elastomers may be formed from divinyl compounds, in particular from polyoxyalkylenes having at least two vinyl groups, reacting with Si—H bonds of a polysiloxane. The polyoxyalkylenated silicone elastomers may be conveyed in the form of a gel composed of an elastomeric organopolysiloxane included in at least one hydrocarbon oil and/or one silicone oil. In these gels, the organopolysiloxane particles are often non-spherical particles.

Polyoxyalkylenated elastomers are described in particular in patents U.S. Pat. No. 5,236,986, U.S. Pat. No. 5,412,004, U.S. Pat. No. 5,837,793, and U.S. Pat. No. 5,811,487, the content of which is incorporated by reference.

As a polyoxyalkylenated silicone elastomer use may be made of those sold under the names “KSG-21”, “KSG-20”, “KSG-30”, “KSG-31”, KSG-32”, “KSG-33”, “KSG-210”, “KSG-310”, “KSG-320”, “KSG-330”, “KSG-340”, and “X-226146” by Shin-Etsu, and “DC9010” and “DC9011” by Dow Corning.

The additional emulsifying silicone elastomer may be present in the composition in an amount ranging from 0.1% to 50% by weight, relative to the total weight of the composition, in particular ranging from 0.1% to 40% by weight, more particularly ranging from 0.5% to 30% by weight, in particular ranging from 0.5% to 20% by weight, and more particularly still ranging from 1% to 10% by weight.

Among the surfactants that may be used in the cosmetic compositions in accordance with the present invention, mention may be made more particularly of hydrophilic organopolysiloxanes other than the silicone polymer described hereinabove.

The hydrophilic radical may correspond to the formula (II): [symbol]
CH₂ [symbol] ₚO [symbol] C₂H₄O [symbol] _{q} [symbol] C₃H₆O [symbol] ᵣX (II)
in which
p ranges from 0 to 5, q ranges from 0 to 100 and r ranges from 0 to 50, with p or q being non-zero,
the units (C₂H₄O) and (C₃H₆O) may be distributed randomly or in blocks, and
X is a hydrogen or a C₁-C₁₀ alkyl radical, where appropriate substituted by one or more functions of hydroxyl, thiol, amine, carboxylic, carboxylate, amide, phosphate, sulphate or sulphonate type.

In particular, p may range from 1 to 5, q from 1 to 100 and r from 1 to 50. X may more particularly feature a hydrogen atom.

In particular, the organopolysiloxane according to the invention may comprise as hydrophilic radical at least one hydroxy-polyalkyleneoxy radical and especially a hydroxy-polyethyleneoxy radical.

The organopolysiloxane according to the invention may especially correspond to the formula (I): 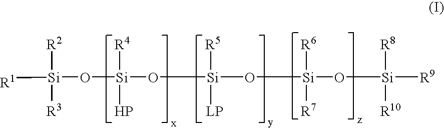
in which:
R¹, R², R³, R⁴, R⁵, R⁶, R⁷, R⁸, R⁹ and R¹⁰ represent, independently of each other, a linear, branched or cyclic, saturated or unsaturated C₁-C₆ alkyl radical,
HP is a radical bearing at least one hydrophilic group as defined hereinabove,
LP is a lipophilic radical, and
x varies from 1 to 5000; y from 0 to 5000; z from 0 to 5000.

As regards the radical LP, it may be selected especially from linear, branched or cyclic C₁-C₄₀ alkyls, organosiloxane groups, fluorine atoms and aryl, aryloxy, C₁-C₄₀ hydrocarbyl acyl and hydroxypropyleneoxy radicals.

According to one particular variant of the invention, the organopolysiloxane belongs to the class of dimethicone-polyethylene glycols and may be selected especially from the group consisting of dimethicone copolyols, in particular cetyl dimethicone copolyol and derivatives thereof. The hydrophilic organopolysiloxane according to the present invention may be the product sold under the brand name Abil WE 09 or Abil EM 90 by the company Degussa-Goldschmidt. The hydrophilic organopolysiloxane according to the present invention may also be the product sold under the reference KF-6017 by the company Shin-Etsu.

The organopolysiloxane compound may be totally or partially fluorinated. In particular, the lower dialkyl siloxy groups may be substituted by one or more fluorine atoms.

The nonionic surfactants more particularly suitable for the cosmetic compositions according to the invention are, in particular, those selected from oxyethylene and/or oxypropylene ethers of glycerol, oxyethylene and/or oxypropylene ethers of fatty alcohols, fatty acid esters of polyethylene glycol, dimethicone copolyol, dimethicone copolyol benzoate, polyoxyalkylenated silicone elastomers, a cyclomethicone/dimethicone copolyol mixture, EO/PO polycondensates, esters and ethers of saccharides, fatty acid esters of polyol, and mixtures thereof.

According to one particular embodiment the surfactant which can be used in the cosmetic compositions in accordance with the present invention is a silicone surfactant selected in particular from dimethicone copolyol, dimethicone copolyol benzoate, dimethicone copolyol phosphates, polyoxyalkylenated silicone elastomers, a cyclomethicone/dimethicone mixture, and mixtures thereof.

Also liable to be suitable for the invention are hydrocarbon-type surfactants exhibiting solubility parameters δd and δa according to the Hansen solubility space that meet the following conditions:
16.2≦δ_{d}≦20 (J/cm³)^{1/2}, preferably 16.3≦δ_{d}≦19 (J/cm³)^{1/2}, and better still 16.9≦_{d}≦18 (J/cm³)^{1/2}, and
9.1≦δₐ≦20 (J/cm³)^{1/2}, preferably 10≦δₐ≦18.1 (J/cm³)^{1/2}, and better still 12≦δₐ≦14.5 (J/cm³)^{1/2}.

The definition of the solubility parameters according to Hansen is well known to those skilled in the art and is described especially in the article by C. M. Hansen: “The three dimensional solubility parameters” J. Paint Technol. 39, 105 (1967). These parameters are also described in document JP-A-08-109 121 from Kao and the document from D. W. van Krevelen “Properties of polymers” (1990), p. 190.

According to this Hansen space:
δ_{d} characterizes the London dispersion forces derived from the formation of dipoles induced during molecular impacts;
δₚ characterizes the Debye interaction forces between permanent dipoles; and
δₙ characterizes the specific interaction forces (such as hydrogen bonding, acid/base, donor/acceptor, etc.).

The parameters δ_{d} δₚ and δₕ are generally expressed in (J/cm³)^{1/2}. They are determined at room temperature (25° C.) and in particular according to the calculation method indicated in the above Kao patent document.

In the composition according to the invention, any fluid, and in particular liquid, hydrocarbon surfactant or mixture of fluid hydrocarbon surfactants that satisfies the above relationships may be used. In this case, the solubility parameters of the mixture are determined from those of the fluid hydrocarbon surfactants taken separately, according to the following relationships:

$${δ}_{Dmixt} = {\sum }_{i} xi {δ}_{Di} ; {δ}_{pmixt} = {\sum }_{i} xi {δ}_{pi} and {δ}_{hmixt} = {\sum }_{i} xi {δ}_{hi}$$

where xi represents the volume fraction of the fluid hydrocarbon surfactant (i) in the mixture.

It is within the capacity of a person skilled in the art to determine the amounts of each fluid hydrocarbon surfactant to obtain a mixture of fluid hydrocarbon surfactants that satisfies the above relationships.

Advantageously, the hydrocarbon surfactant has a chemical structure comprising at least one polar group selected from —COOH; —OH; ethylene oxide 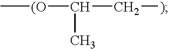
—PO₄—; NHR; NR₁R₂ with R₁ and R₂ possibly forming a ring and representing a linear or branched C₁ to C₂₀ alkyl or alkoxy radical, or 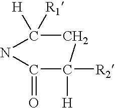
with R₁′ and R₂′ which may be equal to H or to a linear or branched C₁ to C₂₀ alkyl or alkoxy chain.

The hydrocarbon surfactant according to the invention may be selected from:
ether-modified fatty alcohols and in particular the products of addition of ethylene oxide and/or of propylene oxide with i) a linear or branched fatty alcohol or with ii) an alkylphenol,
esters resulting from the reaction of at least one fatty acid with at least one product of addition of ethylene oxide and of glycerol or with at least one product of addition of ethylene oxide and of polyglycerol,
esters resulting from the reaction of glycerol or of polyglycerol with at least one product of addition of ethylene oxide and of a saturated or unsaturated fatty acid,
partial esters resulting from the reaction of at least one linear or branched, saturated or unsaturated fatty acid, of ricinoleic acid or of 12-hydroxystearic acid, with at least one polyol such as glycerol, polyglycerol, pentaerythritol, saccharide alcohols such as sorbitol, and in particular polyglycerol esters,
esters resulting from the reaction of sorbitan with at least one linear or branched, saturated or unsaturated fatty acid,
ether-modified sorbitan esters, and in particular the esters resulting iii) from the reaction of sorbitan with at least one product of addition of ethylene oxide and of a saturated or unsaturated fatty acid, or iv) of the reaction of at least one saturated or unsaturated fatty acid with at least one product of addition of ethylene oxide and of sorbitan,
products of addition of ethylene oxide with castor oil and/or hydrogenated castor oil,
trialkyl phosphates and alkyl mono-, di- and triphosphates, and
mixtures thereof,
   these compounds satisfying the solubility parameters defined above.

The word “ester” as used in the context of the hydrocarbon surfactants that can be used in the anhydrous cosmetic compositions according to the invention means a monoester, a diester, a triester and, more generally, a polyester.

Preferably, the hydrocarbon surfactant is selected from monoesters, diesters and esters resulting from a partial esterification, i.e. the final ester comprises one or more free —OH functions.

Advantageously, the hydrocarbon surfactant is selected from:
the products of addition of 2 to 30 mol of ethylene oxide and/or of 0 to 5 mol of propylene oxide with i) a linear or branched C₈ to C₄₀ and better still C₈ to C₂₂ fatty alcohol or with ii) an alkylphenol,
esters resulting from the reaction of at least one C₈ to C₄₀ and better still C₈ to C₂₂ fatty acid with at least one product of addition of 1 to 30 mol of ethylene oxide and of glycerol or with at least one product of addition of 1 to 30 mol of ethylene oxide and of polyglycerol,
esters resulting from the reaction of glycerol or of polyglycerol with at least one product of addition of 2 to 30 mol of ethylene oxide and of a saturated or unsaturated C₈ to C₄₀ and better still C₈ to C₂₂ fatty acid,
partial esters resulting from the reaction of at least one linear or branched, saturated or unsaturated C₈ to C₄₀ and better still C₈ to C₂₂ fatty acid, of ricinoleic acid or of 12-hydroxystearic acid with glycerol, polyglycerol, pentaerythritol or sorbitol,
esters resulting from the reaction of sorbitan with at least one linear or branched, saturated or unsaturated C₈ to C₄₀ and better still C₈ to C₂₂ fatty acid,
esters resulting iii) from the reaction of sorbitan with at least one product of addition of 2 to 30 mol of ethylene oxide and of a saturated or unsaturated C₈ to C₄₀ and better still C₈ to C₂₂ fatty acid or iv) from the reaction of at least one saturated or unsaturated C₈ to C₄₀ and better still C₈ to C₂₂ fatty acid with at least one product of addition of 2 to 30 mol of ethylene oxide and of sorbitan,
products of addition of 2 to 60 mol of ethylene oxide with castor oil and/or hydrogenated castor oil,
trialkyl phosphates and alkyl mono-, di- and triphosphates, and
mixtures thereof.

The hydrocarbon surfactant is particularly selected from:
myristyl alcohol oxyethylenated with 15 ethylene oxide (or EO) groups (δ_{d}=17.33 (J/cm³)^{1/2} and δₐ=9.28 (J/cm³)^{1/2}),
polyglyceryl-2 monoisostearate oxyethylenated with 5 EO (δ_{d}=17.34 (J/cm³)^{1/2} and δₐ=12.22 (J/cm³)^{1/2}),
polyglyceryl-3 diisostearate (δ_{d}=16.96 (J/cm³)^{1/2} and δₐ=10.4 (J/cm³)^{1/2}),
glyceryl monoisostearate (δ_{d}=16.32 (J/cm³)^{1/2} and δₐ=11.01 (J/cm³)^{1/2}),
polyglyceryl-2 monoisostearate (δ_{d}=17.03 (J/cm³)^{1/2} and δₐ=13.25 (J/cm³)^{1/2}),
polyglyceryl-3 isostearate (δ_{d}=17.38 (J/cm³)^{1/2} and δₐ=14.48 (J/cm³)^{1/2}),
polyglyceryl-4 isostearate (δ_{d}=17.57 (J/cm³)^{1/2} and δₐ=15.37 (J/cm³)^{1/2}),
polyglyceryl-6 monoisostearate (δ_{d}=17.86 (J/cm³)^{1/2}, and δₐ=16.61 (J/cm³)^{1/2}),
polyglyceryl-10 monoisostearate (δ_{d}=18.22 (J/cm³)^{1/2} and δₐ=18.41 (J/cm³)^{1/2}),
polyglyceryl-2 monooleate (δ_{d}=17.14 (J/cm³)^{1/2} and δₐ=13.39 (J/cm³)^{1/2},
sorbitan isostearate (δ_{d}=17.33 (J/cm³)^{1/2} and δₐ=13.56 (J/cm³)^{1/2}),
sorbitan monooleate (δ_{d}=17.32 (J/cm³)^{1/2} and δₐ=13.66 (J/cm³)^{1/2}),
sorbitan monooleate oxyethylenated with 5 EO ((δ_{d}=17.56 (J/cm³)^{1/2} and δₐ=12.47 (J/cm³)^{1/2}), and
mixtures thereof.

Advantageously, the hydrocarbon surfactant is selected from partial esters of polyglycerol and of isostearic acid, partial esters of polyglycerol and of oleic acid and partial esters of sorbitan and of oleic acid, and mixtures thereof.

As hydrocarbon surfactants that may preferably be used in the composition according to the invention, polyglyceryl-2 monoisostearate such as Salacos 41 manufactured or sold by the company Nisshin Oil Mills, polyglyceryl-3 diisostearate such as Lameform TGI manufactured or sold by the company Cognis, polyglyceryl-2 monooleate such as Rylo PG 29 manufactured or sold by the company Danisco Ingredients, or sorbitan monooleate such as Span 80 manufactured or sold by the company Uniqema, and mixtures thereof, may be selected.

According to another particular embodiment the surfactant which can be used in the cosmetic compositions in accordance with the present invention is a surfactant selected from myristyl alcohol oxyethylenated with 15 ethylene oxide (or EO) groups, oxyethylenated polyglyceryl-2 monoisostearate containing 5 EO, polyglyceryl-3 diisostearate, glycerol monoisostearate, polyglyceryl-2 monoisostearate, polyglyceryl-3 isostearate, polyglyceryl-4 isostearate, polyglyceryl-6 monoisostearate, polyglyceryl-10 monoisostearate, polyglyceryl-2 monooleate, sorbitan isostearate, sorbitan monooleate, oxyethylenated sorbitan monooleate containing 5 EO, and mixtures thereof.

Ionic Surfactant

The ionic surfactants employed in the context of the present invention may be anionic or cationic or amphoteric.

By way of representation of anionic surfactants that are suitable for the invention mention may be made more particularly of the following:
C₁₆-C₃₀ fatty acid salts, especially those deriving from amines, such as triethanolamine stearate;
polyoxyethylenated fatty acid salts, especially those deriving from amines or alkali metal salts, and mixtures thereof;
   phosphoric esters and salts thereof, such as “DEA oleth-10 phosphate” (CRODAFOS N 10N from Croda);
sulphosuccinates such as “disodium PEG-5 citrate lauryl sulphosuccinate” and “disodium ricinoleamido MEA sulphosuccinate”;
   alkylamido ethersulphates, monoglyceride sulphates and alkyl ether sulphates such as sodium lauryl ether sulphate;
   alkylglycerylsulphonates, alkylsulphonates, alkylamidesulphonates, alkylarylsulphonates, α-olefinsulphonates and paraffinsulphonates;
isethionates;
acylglutamates such as “disodium hydrogenated tallow glutamate” (Amisoft HS-21 R, sold by Ajinomoto), and mixtures thereof.

Most particularly suitable for the invention is triethanolamine stearate. The latter is generally obtained by simple mixing of stearic acid and triethanolamine.

By way of representation of cationic surfactants mention may be made in particular of the following:
alkylimidazolidiniums such as isostearylethylimidonium ethosulphate,
ammonium salts such as N,N,N-trimethyl-1-docosanaminum chloride (behentrimonium chloride),
optionally polyoxyalkylenated and/or quaternized salts of fatty amines, optionally polyoxyalkylenated and/or quaternized esters of fatty acids and amino alcohols, quaternary ammonium salts such as tetraalkylammonium, alkylamidoalkyltrialkylammonium, trialkylbenzylammonium, trialkylhydroxyalkylammonium, dialkylamidoalkyl-dimethylammonium and alkylpyridinium chlorides or bromides, and imidazoline derivatives.

The compositions according to the invention may also include one or more amphoteric surfactants such as N acyl amino acids, for instance N-alkyl aminoacetates and disodium cocoamphodiacetate and amine oxides such as stearamine oxide or else silicone surfactants such as the dimethicone copolyol phosphates, for instance that sold under the name “Pecosil PS 100” by Phoenix Chemical, and also sulphobetaines, alkylaminoalkyl-betaines, alkylamidoalkylsulphotaines, and imidazolium derivatives such as those of amphocarboxyglycinate or of amphocarboxypropionate.

Generally speaking, the compositions according to the invention may contain from 0.01% to 40% by weight, in particular from 0.1% to 20% by weight or even from 0.5% to 15% by weight, or even from 3% to 10%, or even from 5% to 8% by weight of surfactant, relative to the total weight of the composition.

According to one particular embodiment the continuous phase of the composition may comprise an aqueous (or hydrophilic) phase or a fatty phase, or a mixture of the two.

According to a variant the cosmetic compositions in accordance with the present invention may be presented in the form of an emulsion, in which the silicone polymer of general formula (I), as defined above, may have the function of surfactant.

For the purposes of the present invention the emulsions contain a lipophilic phase and a hydrophilic phase, the latter not systematically being water.

Accordingly, cosmetic compositions in accordance with the invention may be in water-in-oil, oil-in-water, multiple or anhydrous emulsion form.

Thus the cosmetic compositions in accordance with the invention may be in anhydrous emulsion form.

In particular the composition may possess, for example, a continuous fatty phase possibly containing less than 10% by weight of water, in particular less than 5% by weight of water, or even less than 1% by weight of water relative to the total weight of the composition.

The cosmetic compositions according to the invention are advantageously anhydrous: that is, they may contain less than 5%, in particular less than 3%, in particular less than 2%, and more particularly less than 1% of water relative to the total weight of the composition. In that case they may be present in particular in the form of of oily gels, oily liquids, pastes or sticks or else in the form of a vesicle dispersion containing ionic and/or nonionic liquids.

Fatty Phase

The cosmetic compositions in accordance with the invention comprise a fatty phase comprising, in particular, oils and fats which are solid at ambient temperature (20-25° C.) and atmospheric pressure.

An oil is any fatty substance which is in liquid form at ambient temperature (20-25° C.) and at atmospheric pressure. The liquid fatty phase may also, besides the oils, contain other compounds dissolved in the oils, such as gelling and/or structuring agents.

The cosmetic composition according to the present invention may comprise at least one, and in particular at least two, oil(s).

The oil or oils may be present in a proportion of from 0.1% to 99% by weight, in particular of at least 1% to 90% by weight, more particularly from 5% to 70% by weight, especially from 10% to 60% by weight, or even from 20% to 50% by weight, relative to the total weight of the cosmetic composition according to the invention.

The oils suitable for preparing cosmetic compositions according to the invention may be volatile or non-volatile, silicone or non-silicone oils.

For the purposes of the present invention a “volatile oil” is an oil (or non-aqueous medium) which is capable of evaporating on contact with the skin in less than one hour at ambient temperature and atmospheric pressure. The volatile oil is a volatile cosmetic oil which is liquid at ambient temperature, having in particular a non-zero vapour pressure, at ambient temperature and atmospheric pressure, in particular having a vapour pressure ranging from 0.13 Pa to 40 000 Pa (10⁻³ to 300 mm Hg), and preferably ranging from 1.3 Pa to 13 000 Pa (0.01 to 100 mm Hg), and preferentially ranging from 1.3 Pa to 1300 Pa (0.01 to 10 mm Hg).

For the purposes of the present invention a “non-volatile oil” is an oil having a vapour pressure of less than 0.13 Pa. The volatile or non-volatile oils may be hydrocarbon oils, particularly of animal or plant origin, synthetic oils, silicone oils, fluoro oils, or mixtures thereof.

For the purposes of the present invention a “silicone oil” is an oil containing at least one silicon atom, and in particular at least one Si—O group.

A “hydrocarbon oil” is an oil containing principally hydrogen and carbon atoms and optionally oxygen, nitrogen, sulphur and/or phosphorus atoms.

The volatile hydrocarbon oils may be selected from hydrocarbon oils having 8 to 16 carbon atoms, and especially branched C₈-C₁₆ alkanes (also known as isoparaffins), for instance isododecane (also known as 2,2,4,4,6-pentamethylheptane), isodecane, isohexadecane, and, for example, the oils sold under the trade names Isopars® or Pernethyls®.

As volatile oils it is also possible to use volatile silicones, for example volatile linear or cyclic silicones, especially those having a viscosity≦8 centistokes (8×10⁻⁶ m²/s), and having in particular 2 to 10 silicon atoms, and in particular 2 to 7 silicon atoms, these silicones optionally containing alkyl or alkoxy groups having 1 to 10 carbon atoms. As volatile silicone oils which can be used in the invention mention may be made in particular of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

It is also possible to use volatile fluoro oils such as nonafluoromethoxybutane or perfluoromethylcyclopentane, and mixtures thereof.

The fatty phase of the cosmetic compositions according to the present invention may also comprise at least one volatile oil.

According to one particular embodiment the cosmetic compositions according to the invention contain less than 30% by weight, and in particular less than 15%, in particular less than 10%, and more particularly less than 5%, by weight of volatile oil, relative to the total weight of the composition.

According to another embodiment the cosmetic compositions according to the present invention are free from volatile oils.

The fatty phase of the cosmetic compositions according to the present invention may also comprise at least one non-volatile oil.

The non-volatile oils may in particular be selected from hydrocarbon oils, fluorinated where appropriate, and/or silicone oils which are non-volatile.

As non-volatile hydrocarbon oil mention may be made in particular of the following:
hydrocarbon oils of animal origin,
hydrocarbon oils of plant origin, such as phytostearyl esters, for instance phytostearyl oleate, phytostearyl isostearate and lauroyl/octyldodecyl/phytostearyl glutamate (Ajinomoto, Eldew PS203), triglycerides consisting of fatty acid glycerol esters, in which the fatty acids may have varied chain lengths from C₄ to C₂₄, these chains possibly being linear or branched and saturated or unsaturated; these oils are, in particular, heptanoic or octanoic triglycerides, wheatgerm oil, sunflower oil, grapeseed oil, sesame oil, corn oil, apricot oil, castor oil, shea oil, avocado oil, olive oil, soya oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; shea butter; or else caprylic/capric acid triglycerides such as those sold by Stéarineries Dubois or those sold under the names Miglyol 810®, 812® and 818® by Dynamit Nobel;
synthetic esters having 10 to 40 carbon atoms;
linear or branched hydrocarbons of mineral or synthetic origin, such as petroleum jelly, polydecenes, hydrogenated polyisobutene such as parleam, and squalane, and mixtures thereof, and, in particular, hydrogenated polyisobutene;
synthetic esters such as the oils of formula R₁COOR₂ in which R₁ represents the residue of a linear or branched fatty acid containing 1 to 40 carbon atoms and R₂ represents a hydrocarbon chain, particularly a branched hydrocarbon chain, containing 1 to 40 carbon atoms, on condition that R₁+R₂ is ≧10.

The esters may in particular be selected from the esters, in particular, of fatty acids, such as, for example:
cetostearyl octanoate, esters of isopropyl alcohol, such as isopropyl myristate, isopropyl palmitate, ethyl palmitate, 2-ethylhexyl palmitate, isopropyl stearate or isostearate, isostearyl isostearate, octyl stearate, hydroxylated esters such as isostearyl lactate, octyl hydroxystearate, diisopropyl adipate, heptanoates, and especially isostearyl heptanoate, octanoates, decanoates or ricinoleates of alcohols or of polyalcohols, such as propylene glycol dioctanoate, cetyl octanoate, tridecyl octanoate, 2-ethylhexyl 4-diheptanoate and palmitate, alkyl benzoate, polyethylene glycol diheptanoate, propylene glycol di-2-ethylhexanoate and mixtures thereof, C₁₂ to C₁₅ alcohol benzoates, hexyl laurate, esters of neopentanoic acid such as isodecyl neopentanoate, isotridecyl neopentanoate, isostearyl neopentanoate and octyldodecyl neopentanoate, esters of isononanoic acid such as isononyl isononanoate, isotridecyl isononanoate and octyl isononanoate, and hydroxylated esters such as isostearyl lactate and diisostearyl malate;
polyol esters and pentaerythritol esters, such as dipentaerythritol tetrahydroxystearate/tetraisostearate;
esters of diol dimers and of diacid dimers, such as the products Lusplan DD-DA5® and Lusplan DD-DA7®, sold by Nippon Fine Chemical and described in application FR 0302809, filed on 6 Mar. 2003, the content of which is incorporated into the present application by reference;
fatty alcohols which are liquid at ambient temperature, containing a branched and/or unsaturated carbon chain having 12 to 26 carbon atoms, such as 2-octyldodecanol, isostearyl alcohol, oleyl alcohol, 2-hexyldecanol, 2-butyloctanol and 2-undecylpentadecanol;
higher fatty acids, such as oleic acid, linoleic acid, linolenic acid and mixtures thereof; and
dialkyl carbonates, the two alkyl chains possibly being identical or different, such as the dicaprylyl carbonate sold under the name Cetiol CC®, by Cognis.

The non-volatile silicone oils which can be used in the composition according to the invention may be non-volatile polydimethylsiloxanes (PDMS), polydimethylsiloxanes containing alkyl or alkoxy groups, pendent or at the ends of a silicone chain, the groups each having 2 to 24 carbon atoms, phenyl silicones such as phenyl trimethicones, phenyl dimethicones, phenyltrimethylsiloxydiphenylsiloxanes, diphenyl dimethicones, diphenylmethyldiphenyltrisiloxanes, and 2-phenylethyl trimethylsiloxysilicates, dimethicones or phenyl trimethicone with a viscosity of less than or equal to 100 cst, and mixtures thereof.

The non-volatile silicone oil suitable advantageously for formulating cosmetic compositions according to the invention is selected in particular from dimethicones or phenyl trimethicones with a viscosity of less than or equal to 100 cst, and in particular 2 cSt, 6 cSt, 10 cSt and 50 cSt dimethicones.

Advantageously the non-volatile oil present in the cosmetic compositions in accordance with the present invention is selected in particular from hydrogenated polyisobutene, isostearyl heptanoate, isononyl isononanoate, isotridecyl isononanoate, diisostearyl malate, dipentaerythritol tetrahydroxystearate/tetraisostearate, 2-octyldodecanol, and mixtures thereof.

According to one particular embodiment the non-volatile oil present in the cosmetic composition in accordance with the present invention is a mixture of hydrogenated polyisobutene, isostearyl heptanoate, isononyl isononanoate, isotridecyl isononanoate, diisostearyl malate, dipentaerythritol tetrahydroxystearate/tetraisostearate and 2-octyldodecanol.

The non-volatile oils may be present in the compositions according to the invention in an amount ranging from 20% to 99% by weight, in particular from 30% to 80% by weight, and especially from 40% to 80% by weight, relative to the total weight of the composition.

According to one particular embodiment, when the liquid fatty phase of the cosmetic compositions according to the invention is a silicone oil, it is present in an amount ranging from 0 to 90% by weight, in particular from 0.1% to 80% by weight, and especially from 2% to 80% by weight, relative to the total weight of the composition.

According to another embodiment the silicone oil is present in the cosmetic compositions in accordance with the present invention in a weight ratio, relative to the silicone polymer of general formula (I), varying from 80:1, and in particular from 60:1, and more particularly from 40:1.

The liquid fatty phase may where appropriate be thickened, gelled or structured by incorporation therein of a gelling agent for the liquid fatty phase, as defined below.

The compositions according to the invention may also comprise at least one compound selected from waxes, pasty fatty substances and mixtures thereof.

The wax is solid at ambient temperature (25° C.), features a reversible solid/liquid state change, having a melting temperature of greater than 30° C. and possibly ranging up to 200° C., having a hardness of more than 0.5 MPa, and exhibiting in the solid state an anisotropic crystalline organization. It may be hydrocarbon-, fluoro- and/or silicone-based and may be animal, vegetable, mineral or synthetic in origin. It may be selected for example from beeswax, carnauba wax, candelilla wax, paraffin waxes, hydrogenated castor oil, synthetic waxes such as polyethylene waxes (preferably with a molecular weight of between 400 and 600) or Fischer-Tropsch waxes, silicone waxes such as alkyl or alkoxy dimethicones having 16 to 45 carbon atoms, ceresines or ozokerites, such as, for example, isoparaffins whose melting point is less than 40° C., such as EMW-0003, sold by Nippon Seirou, α-olefin oligomers, such as the Performa V® polymers 825, 103 and 260, sold by New Phase Technologies; ethylene-propylene copolymers, such as Performalene® EP 700, and microcrystalline waxes whose melting point is greater than 85° C., such as the Hi-Mice products 1070, 1080, 1090 and 3080, sold by Nippon Seirou, and mixtures thereof.

Advantageously the wax used in the cosmetic compositions in accordance with the invention is selected from polyethylene waxes, candelilla wax and mixtures thereof.

According to one particular embodiment the cosmetic compositions according to the present invention comprise a mixture of polyethylene wax and candelilla wax.

According to one particular embodiment the wax or waxes used in the cosmetic compositions in accordance with the present invention is or are present in an amount varying from approximately 1.5% to approximately 20%, in particular from approximately 3% to approximately 15%, in particular from approximately 5% to approximately 10%, and more particularly from approximately 6.5% to approximately 8.5% by weight relative to the total weight of the composition.

The cosmetic compositions in accordance with the present invention may also comprise at least one pasty compound.

By “pasty” for the purposes of the present invention is meant a fatty compound which exhibits a reversible solid/liquid state change and at a temperature of 23° C. comprises a liquid fraction and a solid fraction. Also meant by pasty is polyvinyl laurate.

The pasty compound for the purposes of the invention advantageously exhibits a hardness at 20° C. ranging from 0.001 to 0.5 MPa, preferably from 0.002 to 0.4 MPa.

Among the pasty compounds which can be used in the composition according to the invention mention may be made of lanolins and lanolin derivatives such as acetylated lanolins, oxypropylenated lanolins or isopropyl lanolate, and mixtures thereof. It is also possible to use esters of fatty alcohols or acids, especially those having 20 to 65 carbon atoms, such as triisostearyl citrate or cetyl citrate; arachidyl propionate; polyvinyl laurate; cholesterol esters such as triglycerides of vegetable origin, for instance hydrogenated vegetable oils, viscous polyesters and mixtures thereof. As a triglyceride of vegetable origin it is also possible to use hydrogenated castor oil derivatives, such as Thixinr® from Rheox.

Mention may also be made of the polyesters resulting from the esterification of a carboxylic acid and an aliphatic hydroxycarboxylic acid ester. For example, Risocast® DA-L (ester obtained from the esterification reaction of hydrogenated castor oil with dilinoleic acid in proportions of 2 to 1) and Risocast® DA-H (ester resulting from the esterification of hydrogenated castor oil with isostearic acid in proportions of 4 to 3), which are sold by the Japanese company Kokyu Alcohol Kogyo.

As pasty compounds suitable advantageously for the formulation of the cosmetic compositions in accordance with the present invention, mention may be made of hydrogenated cocoglycerides.

Mention may also be made of pasty silicone compounds such as the high molecular weight polydimethylsiloxanes (PDMS), and especially those having pendent chains of the alkyl or alkoxy type having 8 to 24 carbon atoms, and a melting point of 20-55° C., such as stearyl dimethicones, particularly those sold by Dow Corning under the trade names DC2503® and DC25514® and mixtures thereof.

Aqueous Phase

According to certain aspects of the present invention the composition according to the invention may comprise at least one aqueous medium, constituting an aqueous phase, which may form the continuous phase of the composition.

The aqueous phase may be composed essentially of water.

It may also comprise a mixture of water and a water-miscible organic solvent (with a miscibility in water of more than 50% by weight at 25° C.), such as lower monoalcohols having 1 to 5 carbon atoms, for instance ethanol, isopropanol, glycols having 2 to 8 carbon atoms, for instance propylene glycol, ethylene glycol, 1,3-butylene glycol and dipropylene glycol, C₃-C₄ ketones and C₂-C₄ aldehydes.

The aqueous phase (water and, optionally, the water-miscible organic solvent) may be present in an amount ranging from 0.1% to 40% by weight, in particular ranging from 0.1% to 20% by weight, and especially 0.1% to 10% by weight, relative to the total weight of the composition.

This aqueous phase may, where appropriate, be thickened, gelled or structured by incorporation therein, moreover, of a conventional aqueous gelling agent, particularly one of mineral origin such as clay, for example, and/or of organic origin, such as an aqueous gelling polymer, as defined below.

Gelling Agents

For the purposes of the present invention a “gelling agent” is a compound capable of structuring the liquid fatty phase of the composition.

By structured liquid phase is meant a stiffened or gelled, or merely thickened, liquid phase.

By stiffened liquid phase is meant that the phase does not flow under its own weight when combined with a gelling or structuring agent.

By gelled or thickened liquid phase is meant that the viscosity of said phase is increased by virtue of its combination with a gelling or structuring agent.

For the purposes of the present invention, the term “non-fluid” qualifies any cosmetic composition lacking the ability to flow under its own weight.

More particularly, this type of cosmetic composition is also qualified by the term “cast composition” in contrast to what are called fluid compositions. These compositions are characterized by a pasty appearance at ambient temperature (20-25° C.).

According to one particular embodiment the gelling agent may be selected in particular from gelling agents in polymeric form and gelling agents in mineral or inorganic form.

For the purposes of the present invention an “inorganic gelling agent” is a gelling agent whose principal structural elements are devoid of carbon atoms. However, these gelling agents may also include carbon derivatives as secondary or modifying structural elements. As an example of such inorganic gelling agents mention may be made of clays modified with fatty acid salts, as described below.

In contrast, in the context of the present invention, organic gelling agents will be defined as being agents whose principal structural elements include at least one carbon atom. As examples of such agents mention may be made of polyorganosiloxanes, silicone gums or polyurethanes, as defined below.

In another embodiment the gelling agent is not soluble in an aqueous phase or in water, and in particular is a liquid-fatty-phase gelling agent.

Viscosity Measurement

The dynamic viscosity of the cosmetic compositions in accordance with the present invention at 25° C. may be measured by means of a rotational viscometer of Mettler RM 180 type. Advantageously it varies from 0.1 to 120 Pa·s.

The Mettler RM 180 instrument (Rhéomat) may be equipped with different spindles as a function of the size order of the viscosity to be measured. For a viscosity varying from 0.18 to 4.02 Pa·s, the instrument is equipped with a spindle 3. For a viscosity varying from 1 to 24 Pa·s, the instrument is equipped with a spindle 4, and for a viscosity varying from 8 to 122 Pa·s the instrument is equipped with a spindle 5.

The viscosity is read off in the instrument in deviation units (DU). Tables supplied with the measuring instrument then allow the corresponding value in poises to be obtained.

The rotational speed of the spindle is approximately 200 revolutions/minute.

When the spindle is set in rotation, at a constant driven rotational speed (in this case 200 revolutions/minute), the viscosity value of the composition may vary over time until it reaches a constant value. Measurements are taken at regular intervals until constant measurement values are obtained. The viscosity value which has become constant over time is the value taken as being the dynamic viscosity value of the composition according to the invention. According to the 75 measurement system, the viscosity measurement is taken after 10 minutes.

The viscosity of the composition varies in particular from 0.5 to 50 Pa·s and more particularly from 3.5 to 2.5 Pa·s.

The addition of gelling agent is one of the formulating elements which is known to the skilled person for the purpose of modifying the viscosity of the composition.

Accordingly, depending on the proportion of aqueous phase and/or liquid-fatty-phase gelling agent present, the cosmetic compositions according to the present invention may be considered as being fluid or solid in nature.

Liquid-Fatty-Phase Gelling Agents

According to one particular embodiment the cosmetic compositions in accordance with the present invention comprise at least one liquid-fatty-phase gelling agent.

According to a specific embodiment the liquid-fatty-phase gelling agent may be an inorganic gelling agent.

According to another specific embodiment the liquid-fatty-phase gelling agent may be a non-crystalline, non-silicone gelling agent.

In one particular embodiment the liquid-fatty-phase gelling agents which can be used in the cosmetic compositions according to the present invention are selected, in particular, from agents which gel via chemical crosslinking and agents which gel via physical crosslinking.

a. Gelling Agents which Gel by Chemical Crosslinking

According to one embodiment use is made advantageously, for preparing cosmetic compositions in accordance with the invention, of crosslinked elastomeric polyorganosiloxanes of three-dimensional structure, such as MQ silicone resins, polyalkylsesquioxanes, especially polymethylsesquioxanes, and resins crosslinked via hydrosilylation. These silicone resins may carry hydrophilic groups, such as polyoxyethylene or copoly(oxyethylene/oxypropylene).

As polyorganosiloxanes which can be used in the invention mention may be made in particular of the crosslinked elastomeric polyorganosiloxanes described in application EP-A-0 295 886, the disclosure content of which is incorporated into this text by reference. These gelling agents include at least one polyorganosiloxane having at least two lower C₂ to C₆ alkenyl groups per molecule; and a polyorganosiloxane having at least two hydrogen atoms bonded to a silicon atom per molecule. It is also possible to use the polyorganosiloxanes described in patent U.S. Pat. No. 5,266,321, the disclosure content of which is likewise incorporated into this text by reference.

In particular the gelling agents which gel by chemical crosslinking are selected from:
polyorganosiloxanes comprising R₂SiO and RSiO_{1.5} units and optionally R₃SiO_{0.5} and/or SiO₂ units, in which the radicals R are selected, independently of one another, from a hydrogen, an alkyl such as methyl, ethyl or propyl, an aryl such as phenyl or tolyl, an unsaturated aliphatic group such as vinyl, the weight ratio of the R₂SiO units to the RSiO_{1.5} units ranging from 1/1 to 30/1;
polyorganosiloxanes which are insoluble and swellable in silicone oil, obtained by addition of a polyorganohydrosiloxane (1) and a polyorganosiloxane (2) having unsaturated aliphatic groups such that the amount of hydrogen or of unsaturated aliphatic groups in (1) and (2) respectively ranges from 1 to 20 mol % when the polyorganosiloxane is non-cyclic and from 1 to 50 mol % when the polyorganosiloxane is cyclic. These polyorganosiloxanes may optionally contain from 1 to 40 oxyalkylene groups, such as oxypropylene and/or oxyethylene groups.

As examples of polyorganosiloxanes which can be used according to the invention mention may also be made of those sold or manufactured under the names KSG6 by Shin-Etsu, Trefil E-505C or Trefil E-506C from Dow Corning, Gransil from Grant Industries (SR-CYC, SR DMF10, SR-DC556) or those sold in the form of preconstituted gels (KSG15, KSG17, KSG16, KSG18 and KSG21 from Shin-etsu, Gransil SR 5CYC gel, Gransil SR DMF 10 gel, Gransil SR DC556 gel, SF 1204 and JK 113 from General Electric.

These gelling agents can be used, in the cosmetic compositions according to the invention, alone or in mixtures.

b. Gelling Agents which Gel via Physical Crosslinking

The liquid-fatty-phase gelling agents may also be selected from gelling agents which act via a physical crosslinking, in particular via molecular agitation, hydrogen interactions or dipolar interactions.

Among all of these gelling agents, fat-soluble polymers having liquid-crystal groups are particularly suitable for the implementation of the present invention.

Gelling agents which gel via molecular agitation are polymers having high molecular weights, greater in particular than 500 000, such as silicone gums.

The silicone gum may correspond to the formula (VIII): 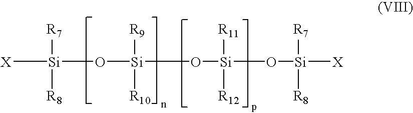
in which:
R₇, R₈, R₁₁ and R₁₂ are, independently of one another, identical or different and each is selected from alkyl radicals containing 1 to 6 carbon atoms,
R₉ and R₁₀ are identical or different, and each is selected from alkyl radicals containing 1 to 6 carbon atoms and aryl radicals,
X is selected from alkyl radicals containing 1 to 6 carbon atoms, a hydroxyl radical and a vinyl radical,
n and p are selected so as to give the silicone gum a viscosity greater than 100 000 mPa·s, such as greater than 500 000 mPa·s.

In particular, n and p may each take values ranging from 0 to 5000, such as from 0 to 3000.

Among the silicone gums which can be used as a gelling agent according to the invention, mention may be made of those for which:
the substituents R₇ to R₁₂ and X represent a methyl group, p=0 and n=2700, such as the product sold or manufactured under the name SE30 by General Electric,
the substituents R₇ to R₁₂ and X represent a methyl group, p=0 and n=2300, such as the product sold or manufactured under the name AK 500 000 by Wacker,
the substituents R₇ to R₁₂ represent a methyl group, the substituent X represents a hydroxyl group, p=0 and n=2700, in the form of a 13% solution in cyclopentasiloxane, such as the product sold or manufactured under the name Q2-1401 by Dow Corning,
the substituents R₇ to R₁₂ represent a methyl group, the substituent X represents a hydroxyl group, p=0 and n=2700, in the form of a 13% solution in polydimethylsiloxane, such as the product sold or manufactured under the name Q2-1403 by Dow Corning, and
the substituents R₇, R₈, R₁₁, R₁₂ and X represent a methyl group and the substituents R₉ and R₁₀ represent an aryl group, such that the molecular weight of the gum is approximately 600 000, for example the product sold or manufactured under the name 761 by Rhône-Poulenc (Rhodia Chimie).

Gelling agents which gel via hydrogen interactions are selected in particular from:
aminosilicone polymers having triazinyl groups or pyrimidinyl groups attached to the amino groups of aminosilicones, as described in patent application EP 0 751 170, the disclosure content of which is incorporated into this text by reference,
non-silicone polyamides whose ends carry ester or triamide functions, such as the compounds described in patents and patent applications U.S. Pat. No. 5,783,657, U.S. Pat. No. 6,268,466, WO 01/95871, WO 00/40216, U.S. 2002/0035237 and EP 1 068 856, the disclosure content of which is incorporated into this text by reference,
polyurethanes, such as the compounds described in patent applications DE 100 22 247 and FR 2 814 365, the disclosure content of which is incorporated into this text by reference, and
(meth)acrylic and/or vinylic polymers which carry side groups able to give rise to mutual hydrogen interactions, such as the compounds described in patent application WO 93/01797, the disclosure content of which is incorporated into this text by reference.

The gelling agents may also be selected from:
copolymers such as the polystyrene-silicone or the polyethylene-silicone described in patents U.S. Pat. No. 6,225,390, U.S. Pat. No. 6,160,054 and U.S. Pat. No. 6,174,968, the disclosure content of each of which is incorporated into this text by reference,
copolymers comprising a silicone sequence and another sequence, or graft, which is polyvinylic or poly(meth)acrylic, such as those described in patents U.S. Pat. No. 5,468,477 and U.S. Pat. No. 5,725,882, the disclosure content of each of which is incorporated into this text by reference,
polymers or copolymers resulting from the polymerization or copolymerization of an ethylenic monomer containing one or more ethylenic bonds, which are preferably conjugated (or dienes),
polymers or copolymers resulting from the polymerization or copolymerization of an ethylenic monomer; in particular it is possible to use vinylic, acrylic or methacrylic copolymers. The ethylenic gelling agent may comprise, for example, a styrene (S) block or an alkylstyrene (AS) block and a block selected from ethylene/butylene (EB), ethylene/propylene (EP), butadiene (B), isoprene (I), acrylate (A), methacrylate (MA) blocks or a combination of these blocks.

In one particular embodiment a copolymer comprising at least one styrene block is used as gelling agent. A triblock copolymer, and in particular those of polystyrene/polyisoprene or polystyrene/polybutadiene type, such as those sold or manufactured under the name “Luvitol HSB” by BASF and those of the polystyrene/copoly(ethylene-propylene) type or, alternatively, those of polystyrene/copoly(ethylene/butylene) type, such as those sold or manufactured under the brand name “Kraton” by Shell Chemical Co. or Gelled Permethyl 99A by Penreco, may be used. Styrene-methacrylate copolymers may also be used.

As an ethylenic gelling agent which can be used in the compositions in accordance with the invention, mention may be made, for example, of Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS), Kraton D-1107 (SIS), Gelled Permethyl 99A-750, Gelled Permethyl 99A-753-58, Gelled Permethyl 99A-753-59, Versagel 5970 and Versagel 5960 from Penreco, and OS 129880, OS 129881 and OS 84383 from Lubrizol (styrene-methacrylate copolymer).

Diblocks or triblocks such as polystyrene-copoly(ethylene/propylene) or polystyrene-copoly(ethylene/butylene), such as those described in patent applications WO 98/38981 and U.S. 2002/0055562, are likewise included in the present invention.

Gelling agents which gel via dipolar interactions are selected in particular from the compounds described in documents WO 01/30886 and U.S. Pat. No. 6,228,967, the disclosure content of each of which is incorporated into this text by reference. The ionized groups of said compounds, for example zwitterionic groups, give rise to said dipolar interactions.

Gelling agents such as fat-soluble polymers having liquid-crystal groups are also suitable for the preparation of cosmetic compositions according to the present invention. In particular, fat-soluble polymers whose skeleton is of silicone, vinylic and/or (meth)acrylic type, and which possess liquid-crystal side groups, are used advantageously, and especially the compounds described in patent application FR 2 816 503, the disclosure content of which is incorporated into this text by reference.

According to another particular embodiment the liquid-fatty-phase gelling agent may be in mineral or inorganic form.

The gelling agent may be a modified clay. As modified clays which can be used mention may be made of hectorites modified with an ammonium chloride of a C₁₀ to C₂₂ fatty acid, such as a hectorite modified with distearyldimethylammonium chloride, also known as quaternium-18 bentonite, such as the products sold or manufactured under the names Bentone 34 by Rheox, Claytone XL, Claytone 34 and Claytone 40 sold or manufactured by Southern Clay, the modified clays known under the name of benzalkonium quaternium-18 bentonites bentonites and sold or manufactured under the names Claytone HT, Claytone GR and Claytone PS by Southern Clay, clays modified with stearyldimethylbenzoylammonium chloride, known as stearalkonium bentonites, such as the products sold or manufactured under the names Claytone APA and Claytone AF by Southern Clay, and Baragel 24, sold or manufactured by Rheox.

As other mineral gelling agents which can be used in the invention, mention may be made of silica, such as pyrogenic silica. The pyrogenic silica may have a particle size which may be nanometric or micrometric, ranging for example from around 5 nm to 200 nm.

Pyrogenic silicas may be obtained by high-temperature hydrolysis of a volatile compound of silicon in an oxyhydrogen flame, producing a finely divided silica. This process allows hydrophilic silicas to be obtained which possess a substantial number of silanol groups on their surface. The silanol groups may be replaced, for example, by hydrophobic groups, thereby giving rise to a hydrophobic silica. The hydrophobic groups may be:
trimethylsiloxyl groups, which are obtained in particular by treating pyrogenic silica in the presence of hexamethyldisilazane. The silicas thus treated are known as “silica silylate” according to the CTFA (6th edition, 1995). They are sold or manufactured, for example, under the references “Aerosil R812®”by Degussa and “Cab-O-Sil TS-530®” by Cabot;
dimethylsilyloxyl or polydimethylsiloxane groups, which are obtained in particular by treating pyrogenic silica in the presence of polydimethyldisiloxane or dimethyldichlorosilane. The silicas thus treated are known as “silica dimethylsilylate” according to the CTFA (6th edition, 1995). They are sold or manufactured, for example, under the references “Aerosil R972®” and “Aerosil R974®” by Degussa and “Cab-O-Sil TS-610®” and “Cab-O-Sil TS-720®” by Cabot;
groups derived from the reaction of pyrogenic silica with silane alkylates or siloxanes. These treated silicas are, for example, the products sold or manufactured under the reference “Aerosil R805®” by Degussa.

According to the invention a hydrophobic silica, such as a pyrogenic silica, may be used as gelling agent.

Generally speaking, the gelling agent may be used, for example, in concentrations ranging from 0.05% to 35% of the total weight of the composition, for example from 0.5% to 20% or from 1% to 10%.

In particular, the liquid-fatty-phase gelling agent, is present in a proportion of from 0.05% to 35% by weight, and in particular from 0.5% to 20% by weight, and more particularly from 1% to 10% by weight, relative to the total weight of the composition.

Advantageously the liquid-fatty-phase gelling agent is present in a weight ratio, relative to the liquid fatty phase to be gelled, at a proportion of 1:90, and in particular of 1:80, in particular of 1:60, in particular of 1:40, in particular of 1:20, and more particularly of 1:10.

According to one particular embodiment the liquid-fatty-phase gelling agent used for structuring the cosmetic compositions according to the invention may be selected from crosslinked elastomeric polyorganosiloxanes of three-dimensional structure, such as MQ silicone resins, polyalkylsesquioxanes, in particular polymethylsesquioxanes and resins crosslinked via hydrosilylation; silicone gums; aminosilicone polymers; non-silicone polyamides; polyurethanes; (meth)acrylic and/or vinylic polymers; polymers or copolymers resulting from the polymerization or copolymerization of an ethylenic monomer and containing one or more ethylenic bonds; polymers or copolymers resulting from the polymerization or copolymerization of an ethylenic monomer, in particular vinylic, acrylic or methacrylic copolymers; triblock copolymers, particularly those of polystyrene/polyisoprene type or of polystyrene/polybutadiene type, or of polystyrene/copoly(ethylene-propylene) type or of polystyrene/copoly(ethylene/butylene) type; styrene-methacrylate copolymers; modified clays such as quaternium-18 bentonite, benzalkonium quaternium-18 bentonites and stearalkonium bentonites; and mixtures thereof.

According to one particular embodiment the liquid-fatty-phase gelling agent used for structuring the cosmetic compositions in accordance with the present invention is, in particular, a non-crystalline, non-silicone gelling agent selected in particular from non-silicone polyamides; polyurethanes; (meth)acrylic and/or vinylic polymers; polymers or copolymers resulting from the polymerization or copolymerization of an ethylenic monomer containing one or more ethylenic bonds; polymers or copolymers resulting from the polymerization or copolymerization of an ethylenic monomer, particularly vinylic, acrylic or methacrylic copolymers; triblock copolymers of polystyrene/polyisoprene or polystyrene/butadiene type, or of polystyrene/copoly(ethylene-propylene) type or of polystyrene/copoly(ethylene/butylene) type; styrene-methacrylate copolymers; and mixtures thereof.

According to another particular embodiment the liquid-fatty-phase gelling agent used for structuring the cosmetic compositions in accordance with the present invention is an inorganic or mineral gelling agent selected in particular from modified clays, especially hectorites modified with an ammonium chloride of a C₁₀ to C₂₂ fatty acid, such as quaternium-18 bentonites, benzalkonium quaternium-18 bentonites and stearalkonium bentonites; pyrogenic silica; and mixtures thereof.

When the composition comprises an aqueous phase it may additionally comprise an aqueous-phase gelling agent.

The gelling agent for the aqueous phase that may be used in the cosmetic compositions according to the invention may in particular be characterized by its capacity to form, in water, above a certain concentration, a gel. This composition may vary widely depending on the nature of the gelling agent in question.

By way of illustration this concentration is between 1% and 2% by weight for an acrylamide/sodium 2-acrylamidomethylpropanesulphonate copolymer in inverse emulsion at 40% in polysorbate 80/I-C16 such as that sold under the name “Simulgel 600” by SEPPIC, and is approximately 0.5% by weight for an AMPS/ethoxylated (25 EO) cetearyl methacrylate copolymer crosslinked with trimethylolpropane triacrylate (TMPTA).

The gelling agent may be able a water-soluble gelling polymer and is therefore present in the aqueous phase of the composition in solubilized form.

More particularly, this aqueous-phase gelling polymer may be selected from:
homopolymers or copolymers of acrylic or methacrylic acid or their salts and their esters, and in particular the products sold under the names “Versicol F” or “Versicol K” by Allied Colloid, “Ultrahold 8” by Ciba-Geigy, and polyacrylic acids of Synthalen K type,
copolymers of acrylic acid and acrylamide, sold in the form of their sodium salt under the names “Reten” by Hercules, the sodium polymethacrylate sold under the name “Darvan N°7” by Vanderbilt, and the sodium salts of polyhydroxycarboxylic acids that are sold under the name “Hydagen F” by Henkel,
polyacrylic acid/alkyl acrylate copolymers of Pemulen type,
AMPS (polyacrylamidomethylpropanesulphonic acid, partially neutralized with ammonia and highly crosslinked) sold by Clariant,
the AMPS/acrylamide copolymers of Sepigel or Simulgel type, sold by SEPPIC,
the AMPS/polyoxyethylenated alkyl methacrylate copolymers (crosslinked or non-crosslinked) of Aristoflex HMS type, sold by Clariant,
and mixtures thereof.

As other examples of water-soluble gelling polymers mention may be made of the following:
proteins, such as proteins of plant origin, for instance wheat proteins and soya proteins; proteins of animal origin such as keratins, for example keratin hydrolysates and sulphonic keratins;
anionic, cationic, amphoteric or nonionic chitin or chitosan polymers;
cellulose polymers such as hydroxyethylcellulose, hydroxypropylcellulose, methylcellulose, ethylhydroxyethylcellulose, carboxymethylcellulose, and quaternized cellulose derivatives;
vinylic polymers, such as polyvinylpyrrolidones, copolymers of methyl vinyl ether and malic anhydride, the copolymer of vinyl acetate and crotonic acid, copolymers of vinylpyrrolidone and vinyl acetate; copolymers of vinylpyrrolidone and caprolactam; polyvinyl alcohol;
polymers of natural origin, optionally modified, such as:
   gum arabics, guar gum, xanthan derivatives, karaya gum;
   alginates and carrageenans;
   glycoaminoglycans, hyaluronic acid and its derivatives;
   shellac resin, gum sandarac, dammar, elemis and copal resins;
   deoxyribonucleic acid;
   mucopolysaccharides such as hyaluronic acid, chondroitin sulphates, and mixtures thereof.

The aqueous-phase gelling polymer is generally present in the composition in an amount sufficient to adjust the rigidity modulus to a value greater than or equal to 5500 Pa, or even greater than or equal to 7000 Pa.

The aqueous-phase gelling polymer may be present in the composition according to the invention in an amount, in terms of solids, ranging from 0.1% to 20% by weight, relative to the total weight of the composition, in particular from 0.5% to 20% by weight, in particular from 2% to 20% by weight, in particular from 5% to 15% by weight, relative to the total weight of the composition.

It will be appreciated that this amount is variable, furthermore, depending on whether said polymer is combined or not with an ionic and/or nonionic surfactant and/or a film former, which are also capable of affecting the consistency of said composition.

Colorants

The cosmetic composition in accordance with the invention may, advantageously, incorporate one or more colouring agents, at least one colorant, organic or inorganic, in particular of the type of pigments or nacres which is conventionally used in cosmetic compositions.

By pigments are meant white or coloured, mineral or organic particles which are insoluble in an aqueous solution and are intended for colouring and/or opacifying the resulting film.

The pigments may be present in a proportion of from 0.01% to 15% by weight, in particular from 0.01% to 10% by weight and especially from 0.02% to 5% by weight, relative to the total weight of the cosmetic composition. As mineral pigments which can be used in the invention mention may be made of titanium oxide, zirconium oxide or cerium oxide and also zinc oxide, iron oxide or chromium oxide, ferric blue, manganese violet, ultramarine blue and chromium hydrate.

The pigment in question may also have a structure which may be, for example, of sericite/brown iron oxide/titanium dioxide/silica type. A pigment of this kind is sold for example under reference Coverleaf NS or JS by Chemicals and Catalysts and has a contrast ratio of around 30.

The colorant may further comprise a pigment having a structure which may be, for example, of the type of silica microspheres containing iron oxide. An example of a pigment having this structure is that sold by Miyoshi under reference PC Ball PC-LL-100 P, this pigment being composed of silica microspheres containing yellow iron oxide.

Among the organic pigments which can be used in the invention mention may be made of carbon black, D & C pigments, lakes based on cochineal carmine, on barium, strontium, calcium or aluminium, or else the diketopyrrolopyrroles (DPP) described in documents EP-A-542669, EP-A-787730, EP-A-787731 and WO-A-96/08537.

By “nacres” are meant coloured particles of any shape, iridescent or non-iridescent, which are produced in particular by certain molluscs in their shell or else are synthesized, and which exhibit a colour effect by optical interference.

The nacres may be selected from nacreous pigments such as titanium mica coated with an iron oxide, mica coated with bismuth oxychloride, titanium mica coated with chromium oxide, titanium mica coated with an organic dye, and nacreous pigments based on bismuth oxychloride. The nacreous pigment may also comprise mica particles superposed on whose surface there are at least two successive layers of metal oxides and/or of organic colorants.

Mention may also be made, as examples of nacres, of natural mica coated with titanium dioxide, with iron oxide, with natural pigment or with bismuth oxychloride.

Among nacres available on the market mention may be made of Timica, Flamenco and Duochrome (based on mica), which are sold by Engelhard, the Timiron nacres sold by Merck, the Prestige mica-based nacres sold by Eckart, and the synthetic-mica-based Sunshine nacres sold by Sun Chemical.

The nacres may more particularly possess a yellow, pink, red, bronze, orangey, brown, gold and/or coppery colour or glint.

By way of illustration of nacres which can be employed in the context of the present invention, mention may be made in particular of the golden nacres sold in particular by Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold in particular by Merck under the name Bronze fine (17384) (Colorona) and Bronze (17353) (Colorona) and by Engelhard under the name Super bronze (Cloisonne); the orange nacres sold in particular by Engelhard under the name Orange 363C (Cloisonne) and Orange MCR 101 (Cosmica) and by Merck under the name Passion orange (Colorona) and Matt orange (17449) (Microna); the brown-hued nacres sold in particular by Engelhard under the name Nu-antique copper 340XB (Cloisonne) and Brown CL4509 (Chromalite); the copper-glint nacres sold in particular by Engelhard under the name Copper 340A (Timica); the red-glint nacres sold in particular by Merck under the name Sienna fine (17386) (Colorona); the yellow-glint nacres sold in particular by Engelhard under the name Yellow (4502) (Chromalite); the gold-glint red-hued nacres sold in particular by Engelhard under the name Sunstone G012 (Gemtone); the pink nacres sold in particular by Engelhard under the name Tan opal G005 (Gemtone); the gold-glint black nacres sold in particular by Engelhard under the name Nu antique bronze 240 AB (Timica), the blue nacres sold in particular by Merck under the name Matte blue (17433) (Microna), the silver-glint white nacres sold in particular by Merck under the name Xirona Silver, and the green-golden pinkish orangey nacres sold in particular by Merck under the name Indian summer (Xirona), and mixtures thereof.

The cosmetic composition according to the invention may also comprise water-soluble or fat-soluble dyes in an amount ranging from 0.01% to 10% by weight, in particular ranging from 0.01% to 5% by weight relative to the total weight of the cosmetic composition. The fat-soluble dyes are, for example, Sudan Red, DC Red 17, DC Green 6, β-carotene, soya oil, Sudan Brown, DC Yellow 11, DC Violet 2, DC orange 5, and quinoline yellow. The water-soluble dyes are, for example, beetroot juice and methylene blue.

The cosmetic composition according to the invention may also include at least one material having a specific optical effect.

This effect is different from a simple, conventional hue effect—that is, the unified and stabilized effect of the kind produced by conventional colorants such as monochromatic pigments, for example. In the sense of the invention, “stabilized” signifies devoid of an effect of variability of colour with the angle of observation or else in response to a temperature change.

For example, this material may be selected from particles having a metallic glint, goniochromatic colouring agents, diffracting pigments, thermochromic agents, optical brighteners, and also fibres, particularly of interference type. It will be appreciated that these different materials may be combined in such a way as to produce the simultaneous manifestation of two effects or even of a novel effect in accordance with the invention.

The metallic-glint particles which can be used in the invention are selected in particular from:
particles of at least one metal and/or of at least one metal derivative;
particles comprising a single-substance or multi-substance, organic or inorganic substrate, at least partly coated with at least one metal-glint layer comprising at least one metal and/or at least one metal derivative, and
mixtures of said particles.

Among the metals that can be present in said particles mention may be made, for example, of Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and their mixtures or alloys (for example bronzes and brasses) are preferred metals.

By “metal derivatives” are meant compounds derived from metals, especially oxides, fluorides, chlorides and sulphides.

By way of illustration of these particles mention may be made of aluminium particles, such as those sold under the names Starbrite 1200 EAC® by Silberline, and Metalure® by Eckart.

Mention may also be made of metallic powders of copper or of alloy mixtures such as references 2844 sold by Radium Bronze, metal pigments such as aluminium or bronze, such as those sold under the names Rotosafe 700 from the company Eckart, the silica-sheathed aluminium particles sold under the name Visionaire Bright Silver from the company Eckart, and the metal alloy particles such as silica-sheathed bronze (copper and zinc alloy) powders sold under the name Visionaire Bright Natural gold from the company Eckart.

The particles in question may be particles comprising a glass substrate, such as those sold by Nippon Sheet Glass under the names Microglass Metashine.

The goniochromatic colouring agent may be selected, for example, from multilayer interference structures and liquid-crystal colouring agents.

Examples of symmetrical multilayer interference structures that may be used in the compositions prepared in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by the company Merck (Darmstadt). By way of example, these pigments may be the pigments of silica/titanium oxide/tin oxide structure sold under the name Xirona Magic by the company Merck, the pigments of silica/brown iron oxide structure sold under the name Xirona Indian Summer by the company Merck and the pigments of silica/titanium oxide/mica/tin oxide structure sold under the name Xirona Caribbean Blue by the company Merck. Mention may also be made of the Infinite Colors pigments from the company Shiseido. Depending on the thickness and the nature of the various layers, different effects are obtained. Thus, with the Fe₂O₃/SiO₂/Al/SiO₂/Fe₂O₃ structure, the colour changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

Examples of pigments with a polymeric multilayer structure that may be mentioned include those sold by the company 3M under the name Color Glitter.

Examples of liquid-crystal goniochromatic particles that may be used include those sold by the company Chenix and also the product sold under the name Helicone® HC by the company Wacker.

Polyols

According to one embodiment the cosmetic compositions in accordance with the invention may also, further, comprise at least one polyol or polyhydric alcohol.

By “polyhydric alcohol” or “polyol” is meant, for the purposes of the present invention, any organic molecule containing at least two free hydroxyl groups.

Polyhydric alcohols suitable advantageously for formulating cosmetic compositions according to the present invention are those having, in particular, 2 to 20 carbon atoms, especially 2 to 10 carbon atoms, and more particularly 2 to 6 carbon atoms.

Advantageously, the polyol may be selected, for example, from glycerol, propylene glycol, butylene glycol, pentylene glycol, hexylene glycol, dipropylene glycol, diethylene glycol, sorbitol, hydroxypropylsorbitol, 1,2,6-hexanetriol; glycol ethers (having in particular 3 to 16 carbon atoms, such as the (C₁-C₄)alkyol ethers of mono-, di- or tripropylene glycol and the (C₁-C₄)alkyl ethers of mono-, di- or triethylene glycol; and mixtures thereof.

Fillers

Advantageously, the cosmetic compositions in accordance with the invention may also comprise fillers, of organic or mineral nature, which especially allow them to be imparted improved stability with regard to exudation.

The term “filler” should be understood as meaning colourless or white, solid particles of any form, which are in an insoluble and dispersed form in the medium of the composition. Mineral or organic in nature, they give body or rigidity to the composition, and/or softness, a matt effect and uniformity to the makeup.

The fillers used in the compositions of the present invention may be of lamellar, globular, spherical or fibrous form or in any other form intermediate between these defined forms.

The fillers according to the invention may or may not be surface-coated, in particular surface-treated with silicones, amino acids, fluoro derivatives or any other substance that promotes the dispersion and compatibility of the filler in the composition.

For the purposes of the present invention, the terms “mineral fillers” and “inorganic fillers” are used interchangeably.

Among the mineral fillers that may be used in the compositions according to the invention, mention may be made of talc, mica, silica, trimethylsiloxysilicate, kaolin, hydroxyapatite, boron nitride, hollow silica microspheres (Silica Beads from Maprecos), glass or ceramic microcapsules, silica-based fillers, for instance Aerosil 200 and Aerosil 300; Sunsphere L-31 and Sunsphere H-31 sold by Asahi Glass; Chemicelen sold by Asahi Chemical; and composites of silica and of titanium dioxide, for instance the TSG series sold by Nippon Sheet Glass, and mixtures thereof.

Among the organic fillers that may be used in the compositions according to the invention, mention, mention may be made of polyamide powder (Nylon® Orgasol from Atochem), poly-b-alanine powder and polyethylene powder, polytetrafluoroethylene (Teflon®) powders, lauroyllysine, starch, powders of tetrafluoroethylene polymers, hollow polymer microspheres such as Expancel (Nobel Industrie), precipitated calcium carbonate, magnesium carbonate, magnesium hydrocarbonate, metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate, and Polypore® L 200 (Chemdal Corporation), silicone resin microbeads (for example Tospearl® from Toshiba), crosslinked polyurethane powders comprising a copolymer, said copolymer comprising trimethylol hexyllactone. In particular, it may be a hexamethylene diisocyanate/trimethylol hexyllactone polymer. Such particles are especially commercially available, for example, under the name Plastic Powder D-400® or Plastic Powder D-800® from the company Toshiki, and mixtures thereof.

The fillers may be present in the cosmetic compositions in accordance with the invention in a proportion of from 0.001% to 35% and preferably 0.5% to 15% of the total weight of the composition.

The filler may be, for example, a filler with a mean particle size of less than 100 μm, especially between 1 and 50 μm, for example between 4 and 20 μm.

According to one particular embodiment, the composition according to the invention comprises at least one filler that is present in a proportion of from 0.01% to 60% of the total weight of the composition, in particular from 0.5% to 20% and more particularly from 1% to 10% by weight relative to the total weight of the composition.

Additives

The cosmetic compositions according to the invention may also comprise any additive commonly used in the field in question, selected from film formers as described in application FR 0450540, filed on 18 Mar. 2004, the content of which is incorporated into the present application by reference, and, where appropriate, film forming agents, antioxidants, surfactants such as those described in application FR 2834452, published on 11 Jul. 2003, the content of which is incorporated into the present application by reference, essential oils, preservatives, perfumes, neutralizing agents, moisturizers, antiseptics, vitamins such as vitamin B3, vitamin E or a derivative thereof, and UV protectants.

Of course, the person skilled in the art will take care to select the optional additive(s) added to the cosmetic composition according to the invention in such a way that the advantageous properties intrinsically attaching to the composition in accordance with the invention are not, or not substantially, adversely affected by the envisaged addition.

According to one particular version, the silicone polymer of general formula (I) used in the cosmetic compositions in accordance with the present invention is selected from polyglyceryl-3 polydimethylsiloxyethyl dimethicone, lauryl polyglyceryl-3 polydimethylsiloxyethyl dimethicone, polyglyceryl-3 disiloxane dimethicone, and mixtures thereof.

According to another version the silicone polymer of general formula (I) used in the cosmetic compositions in accordance with the present invention is selected from the silicone polymers sold by Shin-Etsu under references KF 6100®, KF 6104®, KF 6105®, and mixtures thereof.

According to another version the cosmetic composition according to the invention combines polyglyceryl-3 polydimethylsiloxyethyl dimethicone and, as film former, the acrylate/stearyl acrylate/dimethicone methacrylate copolymer, particularly that sold under reference KP 561® by Shin-Etsu.

According to yet another version the cosmetic composition according to the invention combines polyglyceryl-3 polydimethylsiloxyethyl dimethicone and at least one wax, selected in particular from polyethylene waxes, candelilla wax, hydrogenated cocoglyceride wax, and mixtures thereof.

It is apparent, obviously, that certain components which can be used in the cosmetic compositions according to the present invention may belong at one and the same time to different classes of compounds. Accordingly, it is not outside the bounds of customary work of the person skilled in the art to adjust the amount of a compound belonging to different classes such that its presence in the formulation is translated into the desired effect, and corresponds, where appropriate, to the effect obtainable by the presence of compounds belonging to said different classes.

The cosmetic composition according to the invention may be in the form, in particular, of a lip makeup product, in particular a lipstick, or a lip balm.

The examples of compositions below are given by way of illustration and without any limitative effect on the invention.

Staying Power of the Composition

The “staying power” denotes the property of the cosmetic composition according to the invention to transfer to a lesser extent onto objects with which it may come into contact, and the property of withstanding interaction with liquids, such as tears or perspiration, or contact with foods during a meal in the case of a lipstick, for example, and the property of not migrating beyond the initial outline of the makeup, particularly in the case of lipsticks, into the wrinkles and fine lines around the lips.

The cosmetic composition according to the invention exhibits the advantage of not transferring, at least partially, in other words of leaving only minor traces on certain substrates with which it may come into contact, and in particular a glass, a cup, a cigarette, a handkerchief, an item of clothing, or the skin. The transfer of cosmetic compositions gives rise to poor staying power of the applied film, making it necessary to renew application of the composition regularly.

The cosmetic composition according to the invention further exhibits good permanence of its colour. The film of composition applied to the skin, lips and/or epidermal derivatives may generally be impaired during contact with liquids, especially water or beverages consumed, for example, during a meal, or alternatively oils, such as food oils or else sebum or else saliva. The colour permanence may thus be characterized by the colourfastness to water and/or the colourfastness to oil.

Hence the evaluation of the staying power properties of the cosmetic composition according to the invention may be characterized by measurement of at least one of the four following parameters: transfer resistance, colourfastness to water, colourfastness to oil, and migration resistance.

The parameters of transfer resistance, colourfastness to water, and colourfastness to oil may be measured in succession according to the protocol described below.

The measurements are performed, for example, on the inner surface of the forearm, which has been washed and allowed to dry naturally at ambient temperature for 5 minutes. The cosmetic composition to be tested, a lipstick for example, is applied to three areas of the inner surface of the forearm. The surface area of skin on which the measurements are performed must be at least greater than 1 cm². The measurements are generally made on circular areas with a diameter of approximately 3 cm.

It is necessary for approximately the same amount of cosmetic composition to be applied to each of the three areas. This can be verified by measuring the weight of the cosmetic composition, the lipstick for example, following each of the applications, or by preparing equivalent amounts of sample to be tested beforehand. Generally speaking, for a surface area of 1 cm², an amount equal to approximately 2 mg is required (if the surface area has a diameter of 3 cm, then an amount of approximately 28 mg is required).

Following application of the cosmetic composition, the colour, L₁*a₁*b₁*, is measured in each of the three areas, and the average value obtained corresponds to the initial colour of the composition. The colour measurement may be performed using a Minolta colorimeter of the CR200 or CR300 or CM500 or CM1000 or CM2000 series. The Minolta series CR200 colorimeter is used in particular.

20 mg/cm² of water are added to each of the three areas to be tested (for surface areas with a diameter of approximately 3 cm, approximately 280 mg of water must be applied). Each of the areas to be tested is then massaged manually for a few seconds, in particular from 2 to 5 seconds, and more particularly 2 seconds.

One thickness of a commercial white-paper handkerchief such as Kleenex, the L₀*a₀*b₀* colour of which has been measured, is applied to each made-up area for approximately 5 seconds and at a force of approximately 100 g/f, which may be applied using a digital pressure dynamometer DPZ-5N from the manufacturer Imada Co. Ltd.

The transfer value T is obtained by subtracting the colour of the white fabric measured before application to the area to be tested, L₀*a₀*b₀*, and the average colour L₂*a₂*b₂* corresponding to the mean of the values obtained for each handkerchief after applying them to each area of the forearm covered with composition to be tested.

A determination is then made of the colour difference ΔE(T) between the colour of the handkerchief before and after application to the area of the forearm bearing the composition:
ΔE(T)=\sqrt{(L₂*−L₀*)²+(a₂*−a₀*)²+(b₂*−b₀*)²}

The lower the resulting ΔE(T) value, the more the cosmetic composition is considered to have a good level of transfer resistance.

Advantageously the cosmetic compositions, according to the present invention, possess a transfer value ΔE(T) between 0 and 45, and in particular less than or equal to 45, in particular less than or equal to 40, especially less than or equal to 35.

Subsequently a measurement is made of the average colour L₃*a₃*b₃* of the composition following application of the handkerchief.

The colourfastness to water may be obtained after the transfer test has been carried out. It is equal, for example, to the colour difference between the average initial colour L₁*a₁*b₁* of the composition applied to the forearm and the average colour L₃*a₃*b₃* of the area of the forearm bearing the composition following application of water and the handkerchief
ΔE(E)=\sqrt{(L₃*−L₁*)²+(a₃*−a₁*)²+(b₃*−b₁*)²}

The lower the resulting value, the more the cosmetic composition is considered to possess good colourfastness to water.

Advantageously, the colourfastness value varies from 0 to 15. Advantageously the cosmetic compositions according to the present invention possess a colourfastness to water of less than or equal to 15, in particular less than or equal to 10, and more particularly less than or equal to 6.

The test for colourfastness to oil is conducted by applying, to the areas to be tested, approximately 20 mg/cm² of food-type oil on each area of the forearm (rapeseed oil, soya oil or sunflower oil), followed by manual massaging for a few seconds, in particular from 2 to 5 seconds, and more particularly for 2 seconds. One thickness of a commercial white-paper handkerchief such as a Kleenex handkerchief is then applied to the area for approximately 5 seconds and at a force of approximately 100 g/f, which may be applied using a digital pressure dynamometer DPZ-5N from the manufacturer Imada Co. Ltd.

The colourfastness to oil, H, is equal to the difference between the average colour L₄*a₄*b₄* of the composition remaining on the forearm after massaging with oil and application of the handkerchief, and the average colour L₁*a₁*b₁* measured initially,
ΔE(H)=\sqrt{(L₄*−L₁*)²+(a₄*−a₁*)²+(b₄*−b₁*)²}

The test for colourfastness to oil is a test that makes it possible, in particular, to evaluate the hold of a cosmetic composition such as a lipstick during a meal.

In particular the silicone polymer is such that, when present in sufficient amount in the composition, the colourfastness to oil of a deposit of said composition, once it has been spread onto a substrate, is less than or equal to 25. Advantageously the cosmetic compositions, according to the present invention, possess a colourfastness to oil of less than or equal to 25, less than or equal to 10, or less than or equal to 8.

Advantageously the silicone polymer is such that, when present in sufficient amount in the composition, the transfer value of said composition, once it has been spread onto a substrate, is less than or equal to 35. The composition preferably has a colourfastness to water of less than 6 and a colourfastness to oil of less than or equal to 8.

Average Gloss of the Composition

Advantageously the silicone polymer of general formula (I) is such that, when present in sufficient amount in the cosmetic composition, the average gloss at 60° of a deposit of said composition, once it has been spread onto a substrate, is greater than or equal to 30 over 100.

The term “average gloss” denotes the gloss as may be measured using a gloss meter, conventionally, by the following method.

A Minolta GM268 gloss meter may be used. The measurements are performed on test areas with a surface area of greater than 1 cm². In standard manner, the surface areas over which the cosmetic compositions to be tested are spread measure approximately 2.5 cm×4 cm.

The cosmetic composition for evaluation is applied to a synthetic surface, of Bioskin type. The amount of cosmetic composition to be applied is approximately 1 mg/cm².

The gloss value is obtained by measuring the reflectance at an angle of approximately 60°.

Five measurements per sample are required; the two measurements having the highest values and the measurement having the lowest value are discarded, and a mean is taken of the remaining three measured values.

The average gloss of the cosmetic compositions according to the present invention is advantageously greater than or equal to 30, in particular greater than or equal to 40, and more particularly greater than or equal to 45.

Comfort

The comfort of the cosmetic composition according to the invention is evaluated by the test described below. In this test, the comfort of the composition is measured by a pulling test on a latex strip. This test predicts the capacity, for a deposition of cosmetic composition, to withstand the flaking and peeling liable to occur in the wake of the movements of the skin.

Samples of cosmetic composition are applied to surface areas of, for example, 2.54×2.54 cm of a latex strip 2.54 cm wide, obtained, for example, by cutting from the wrist area of a glove of Ansell Edmond Industrial Technicians type REF#390, size 9.

The amount of cosmetic composition to be deposited is such that the weight, in terms of solids, of the composition should be approximately 20 mg.

The cosmetic composition is applied to the latex strip using a disposable lip brush, of the type, for example, produced by Femme Cosmetics, Inc., L.A.

The samples thus prepared are left for 24 hours at ambient temperature.

Subsequently the weight of the latex strip containing the deposit of cosmetic composition is measured (B). Subtracting the value of the weight of the latex strip minus cosmetic composition (A) from the value thus measured (B) should correspond to the weight of dry film, and should therefore be approximately 20±2 mg.

The strip of latex bearing the samples of cosmetic composition to be tested is subsequently stretched such that the area supporting the sample should reach a length of approximately 4.445 cm.

The film fragments of the cosmetic composition that have undergone detachment from the strip of latex are observed and then removed by brushing using the lip brush.

The weight of the strip of latex comprising the remaining cosmetic composition is then measured (D).

The percentage weight loss of the film of the cosmetic composition is then calculated by means of the following equation:
Comfort index=[(D−A)/(B−A)]×100.

The measurements are repeated three times for each cosmetic composition tested. The comfort index of the composition according to the invention is equal to the mean of these three measurements.

Advantageously the silicone polymer of general formula (I) is such that, when present in sufficient amount in the cosmetic composition, the comfort index of a deposit of said composition, once it has been spread onto a substrate, is greater than or equal to 90 over 100, preferably greater than 95 over 100.

### EXAMPLE 1

### Lipstick

A lipstick is prepared comprising the polyglyceryl-3 polydimethysiloxyethyl dimethicone sold under reference KF6104® by Shin-Etsu, and whose composition is specified in Table I.

TABLE I

| | Percentages |
|---|---|
| | by mass |
| Hydrogenated polyisobutene | 4 |
| (Parleam HV from NOF) | |
| Isononyl isononanoate | 12 |
| 2-Octyldodecanol | 4.5 |
| Diisostearyl malate | 33.9 |
| Triglycerides of lauric/palmitic/cetyl/stearic | 4 |
| (50/20/10/10) acids (Softisan 100 ® from Sasol) | |
| N-lauroyl-L-lysine | 1 |
| Acrylates/stearyl acrylate/dimethicone methacrylate | 4 |
| copolymer (KP 561 P ® from Shin-Etsu) | |
| Dimethicone 6 cSt (KF 96) | 4 |
| Polyglyceryl-3 polydimethylsiloxyethyl | 13 |
| dimethicone (KF6104 ® from Shin-Etsu) | |
| Preservative | qs |
| Polyethylene wax (NW 500) | 6.6 |
| Polyethylene wax (MW 400) | 3.8 |
| Hydrophobia pyrogenic silica surface-treated | 2 |
| with dimethylsilane (Aerosil R 972 ® from Degussa) | |
| Pigments | 7.0 |
| Simethicone (Antifoam C ® from Dow Corning) | 0.2 |
| Total | 100 |

Procedure

An oily phase is prepared by mixing, with heating (approximately 95° C.), the isononyl isononanoate, the 2-octyldodecanol and the diisostearyl malate with the polyglyceryl-3 polydimethylsiloxyethyl dimethicone and the dimethicone oil. The oily phase thus prepared is stirred at approximately 95° C. and the fillers (N-lauroyl-L-lysine and pyrogenic silica) are added to the mixture.

The waxes, the pigments in the form of a pigment paste, the hydrogenated polyisobutene and the simethicone are then added to the mixture.

The mixture thus obtained is subsequently poured into a lipstick mould and left to cool until a solid composition is obtained.

Thereafter the staying power, comfort and gloss of this composition are measured in accordance with the protocols described earlier on in the description.

Also measured, using the same methods, are the staying power and gloss of two commercial products:
Control A: Jelly Plumpy sold by Maybelline, containing phenyl trimethicone as silicone polymer;
Control B: Aube Rouge Glacé sold by Kao and containing the glycerolated silicone of formula below as silicone polymer

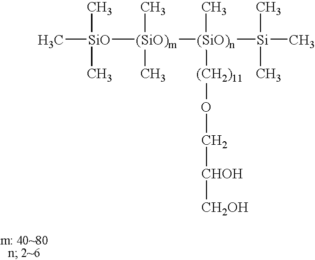

The results obtained are reported in Table II below.

TABLE II

| | Water | Oil | | Comfort | Average |
|---|---|---|---|---|---|
| Formula | resistance | resistance | Transfer | index | gloss |
| Control A | 14.4 | 23.4 | 47.3 | | 42.5 |
| Control B | 7.0 | 8.5 | 42.0 | 85.6 | 48.2 |
| Invention | 5.9 | 7.9 | 32.1 | 99.5 | 48.3 |

In these measurements the colour of the inner surface of the forearm is such that L*=63.9, a*=8.4, b*=13.3, and the colour of the paper tissue is L*=97.9, a*=0.6 and b*=3.3.

The lipstick in accordance with the invention has a better staying power and better comfort than the commercial prior-art products A and B, for an equivalent or even equal gloss.

Moreover, the lipstick in accordance with the invention migrates three to four times less than the prior-art control B.

### EXAMPLES 2 AND 3

### Lipsticks

The percentages are by weight

| | Example 2 | Example 3 |
|---|---|---|
| Hydrogenated polyisobutene | | 4 |
| (Parleam HV from NOF) | | |
| Hydrogenated polyisobutene | 6 | |
| (Parleam Lite from NOF) | | |
| Isononyl isononanoate | 8 | 12 |
| 2-Octyldodecanol | 4.5 | 4.5 |
| Diisostearyl malate | 33.9 | 23.9 |
| Polyglyceryl-2 diisostearate | | 5 |
| (Cosmol 42 V from Nishin Oil) | | |
| Polyglyceryl-2 triisostearate | | 4 |
| (Cosmol 43 N from Nishin Oil) | | |
| Sorbitan sesquioleate | | 1 |
| (Cosmol 82 from Nishin Oil) | | |
| Triglycerides of | 3 | 4 |
| lauric/palmitic/cetyl/stearic | | |
| (50/20/10/10) acids | | |
| (Softisan 100 ® from Sasol) | | |
| N-lauroyl-L-lysine | 1 | 1 |
| Polyvinyl laurate | 3 | |
| Acrylates/stearyl | 2 | 4 |
| acrylate/dimethicone | | |
| methacrylate copolymer | | |
| (KP 561 P ® from Shin-Etsu) | | |
| Dimethicone 6 cSt (KF 96) | 5 | 4 |
| Polyglyceryl-3 | 13 | 13 |
| polydimethylsiloxyethyl | | |
| dimethicone | | |
| (KF6104 ® from Shin-Etsu) | | |
| Preservative | qs | |
| Polyethylene wax (MW 500) | 5.5 | 6.6 |
| Polyethylene wax (MW 400) | 1.1 | |
| Candelilla wax | 4.8 | |
| Microcrystalline wax | | 3.8 |
| Hydrophobic pyrogenic silica | 2 | 2 |
| surface-treated with | | |
| dimethylsilane | | |
| (Aerosil R 972 ® from Degussa) | | |
| Pigments | 7.0 | 7 |
| Simethicone | 0.2 | 0.2 |
| (Antifoam C ® from Dow Corning) | | |
| Total | 100 | 100 |

What is claimed is:

1. An anhydrous cosmetic composition comprising at least one liquid fatty phase, at least one gelling agent for said liquid fatty phase, and at least one silicone polymer of general formula (I):
R¹ₐR²_{b}R³_{c}SiO_{(4-a-b-c)/2} (I)
in which:
a is in a range of from about 1 to about 2.5,
b and c each are independently in a range of from about 0.001 to about 1.5,
R¹, which may be identical or different at each occurrence, is chosen from:
C₁ to C₃₀ alkyl radicals,
C₁ to C₃₀ alkyl radicals, substituted by one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is chosen from C₁ to C₃₀ hydrocarbon radicals and radicals R⁵—(CO)—, in which R⁵ is chosen from C₁ to C₃₀ hydrocarbon radicals,
d is an integer in a range of from 0 to about 15, and
e and f are each integers that are independently in a range of from 0 to about 50,
and combinations thereof,
R² is a radical represented by the general formula (III):
-Q-O—X (III)
wherein:
Q is chosen from divalent C₂ to C₂₀ hydrocarbon radicals that can include at least one ether bond and/or at least one ester bond,
and R³ is an organosiloxane group of general formula (IV): 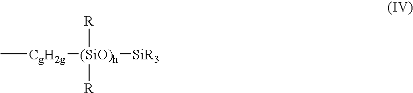
wherein:
radicals R are each radicals independently chosen from the group consisting of from C₁ to C₃₀ alkyl radicals, C₁ to C₃₀ alkyl radicals substituted by one or more fluorine atoms, and aryl and aralkyl radicals,
g is an integer in a range of from 1 to about 5, and
h is an integer in a range from 0 to about 500,
and wherein said gelling agent is a non-crystalline, non-silicone gelling agent.

2. A cosmetic composition comprising at least one colorant, at least one inorganic gelling agent and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is chosen from:
C₁ to C₃₀ alkyl radicals,
C₁ to C₃₀ alkyl radicals substituted by one or more fluorine atoms, amino and/or carboxyl groups,
aryl and aralkyl radicals, and
radicals of general formula (II):
—C_{d}H_{2d}—O—(C₂H₄)ₑ(C₃H₆O)_{f}R⁴ (II)
wherein:
R⁴ is chosen from C₁ to C₃₀ hydrocarbon radicals and radicals R⁵—(CO)—, in which R⁵ is chosen from C₁ to C₃₀ hydrocarbon radicals, and
d is an integer in a range of from 0 to about 15,
e and f are each integers that are independently in a range of from 0 to about 50,
and combinations thereof,
R² is a radical represented by general formula (m):
-Q-O—X (III)
wherein:
Q is chosen from divalent C₂ to C₂₀ hydrocarbon radicals that can include at least one ether bond and/or at least one ester bond, and
X is chosen from polyhydroxylated hydrocarbon radicals,
R³ is an organosiloxane of general formula (IV): 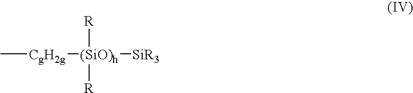
wherein:
radicals R are each radicals independently chosen from C₁ to C₃₀ alkyl radicals, C₁ to C₃₀ alkyl radicals substituted by one or more fluorine atoms, aryl and aralkyl radicals,
g is an integer in a range of from 1 to about 5, and
h is an integer in a range of from 0 to about 500.

3. An anhydrous cosmetic composition comprising at least one liquid fatty phase, at least one gelling agent for said liquid fatty phase, and at least one silicone polymer of general formula (I):
R¹ₐR²_{b}R³_{c}SiO_{(4-a-b-c)/2} (I)
in which:
a is in a range of from about 1 to about 2.5
b and c are each independently in a range of from about 0.001 to about 1.5,
R¹, which may be identical or different at each occurrence, is chosen from:
C₁ to C₃₀ alkyl radicals,
C₁ to C₃₀ alkyl radicals substituted by one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ are chosen from C₁ to C₃₀ hydrocarbon radicals and radicals R⁵—(CO)—, in which R⁵ is chosen from C₁ to C₃₀ hydrocarbon radicals, and
d is an integer in a range of from 0 to about 15,
e and f are each integers that are independently in a range of from 0 to about 50,
and combinations thereof,
R² is a radical of general formula (III):
-Q-O—X (m)
wherein:
Q is a radical chosen from divalent C₂ to C₂₀ hydrocarbon radicals that can include at least one ether bond and/or at least one ester bond, and
X is a polyhydroxylated hydrocarbon radical,
wherein when R² is a group of general formula (IIIA):
—C₃H₆—O[CH₂CH(OH)CH₂O]ₙH (IIIA)
in which n is an integer in a range of from 1 to about 5, R¹ is not a C₁₂ alkyl radical,
and R³ is an organosiloxane of general formula (IV): 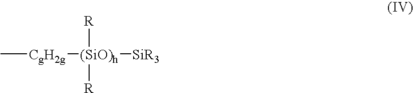
wherein:
radicals R are each radicals independently chosen from the group consisting of C₁ to C₃₀ alkyl radicals, C₁ to C₃₀ alkyl radicals substituted by one or more fluorine atoms, and aryl and aralkyl radicals,
g is an integer in a range of from 1 to about 5,
h is an integer in a range of from 0 to about 500.

4. An anhydrous cosmetic composition comprising at least one liquid fatty phase that comprises at least one silicone oil, at least one gelling agent for said liquid fatty phase, and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is chosen from:
C₁ to C₃₀ alkyl radicals,
C₁ to C₃₀ alkyl radicals substituted by one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from C₁ to C₃₀ hydrocarbon radicals and R⁵—(CO)—, in which R⁵ is chosen from C₁ to C₃₀ radicals, and
d is an integer in a range of from 0 to about 15, and
e and f are each integers that are independently in a range of from 0 to about 50,
and combinations thereof,
R² is a radical of general formula (III):
-Q-O—X (III)
wherein:
Q is chosen from divalent C₂ to C₂₀ hydrocarbon radicals that can include at least one ether bond and/or at least one ester bond, and
X is chosen from substituted polyhydroxy hydrocarbon radicals,
R³ is an organosiloxane of general formula (IV): 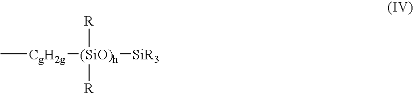
wherein:
groups R are each radicals independently chosen from the group consisting of C₁ to C₃₀ alkyl radicals, C₁ to C₃₀ alkyl radicals substituted by one or more fluorine atoms, aryl and aralkyl radicals,
g is an integer in a range of from 1 to about 5, and
h is an integer in a range of from 0 to about 500.

5. A cosmetic composition comprising at least one liquid fatty phase, at least one gelling agent for said liquid fatty phase, and at least one silicone polymer of general formula (I):
R¹ₐR²_{b}R³_{c}SiO_{(4-a-b-c)/2} (I)
in which:
a is in a range of from about 1 to about 2.5,
b and c each independently are in a range of from about 0.001 to about 1.5,
R¹, which may be identical or different at each occurrence, is chosen from:
C₁ to C₃₀ alkyl radicals,
C₁ to C₃₀ alkyl radicals substituted by one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is chosen from C₁ to C₃₀ hydrocarbon radicals and R⁵—(CO)—, in which R⁵ is chosen from C₁ to C₃₀ radicals,
d is an integer in a range of from 0 to about 15, and
e and f are each integers independently in a range of from 0 to 50,
and combinations thereof,
R² is a radical of general formula (III):
-Q-O—X (III)
wherein:
Q is chosen from divalent C₂ to C₂₀ hydrocarbon radicals that can include at least one ether bond and/or at least one ester bond, and
X is chosen from substituted polyhydroxy hydrocarbon radical,
R³ is an organosiloxane of general formula (IV): 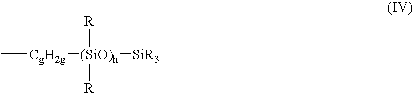
wherein:
radicals R are each radicals independently chosen from the group consisting of C₁ to C₃₀ alkyl radicals, C₁ to C₃₀ alkyl radicals substituted by one or more fluorine atoms, aryl and aralkyl radicals,
g is an integer in a range of from 1 to about 5, and
h is an integer in a range of from 0 to about 500,
and at least one surfactant.

6. The cosmetic composition according to claim 1, wherein the silicone polymer is a compound of general formula (I) in which R¹ is a C₁ to C₁₀.

7. The cosmetic composition according to claim 1, wherein the silicone polymer is a compound of general formula (I):
R¹ₐR²_{b}R³_{c}SiO_{(4-a b-c)/2} (I)
in which:
a is in a range of from about 1 to about 1.4,
b and c are each independently in a range of from about 0.02 to about 0.04, and
R¹ is a C₁ to C₁₀ alkyl radical,
R² is represented by the formula (IIIA):
—C₃H₆O[CH₂CH(OH)CH₂O]ₙH (IIIA)
in which m is in a range of from about 1 to about 5, and
R³ is represented by the formula (IVA):
—C₂H₄(CH₃)₂SiO[(CH₃)₂SiO]ₘSi(CH₃)₃ (IVA)
in which m is in a range of from about 3 to about 9, and
g is an integer in a range of from 1 to about 5, and
h is an integer in a range of from 0 to about 500.

8. The cosmetic composition according to claim 7, wherein the silicone polymer is a compound of general formula (I):
R¹ₐR²_{b}R³_{c}SiO_{(4-a-b-c)/2} (I)
in which:
a is in a range of from about 1 to about 1.4,
b and c are each independently in a range of from about 0.02 to about 0.04,
R¹ is a methyl radical,
R² is represented by the formula (IIIA):
—C₃H₆—O[CH₂CH(OH)CH₂O]ₙH (IIIA)
in which n is in a range of from about 1 to about 5, and
R³ is represented by the formula (IVA):
—C₂H₄(CH₃)₂SiO[(CH₃)₂SiO]ₘSi(CH₃)₃ (IVA)
in which m is in a range of from about 3 to about 9.

9. The cosmetic composition according to claim 3, wherein said liquid-fatty-phase gelling agent is chosen from the group consisting of crosslinked elastomeric polyorganosiloxanes of three-dimensional structure; polyalkylsesquioxanes; silicone gums; aminosilicone polymers; non-silicone polyamides; polyurethanes; (meth)acrylic and/or vinylic polymers; polymers or copolymers resulting from the polymerization or copolymerization of an ethylenic monomer and containing one or more ethylenic bonds; polymers or copolymers resulting from the polymerization or copolymerization of an ethylenic monomer; triblock copolymers of polystyrene/polyisoprene type or of polystyrene/polybutadiene type, or of polystyrene/copoly(ethylene-propylene) type or of polystyrene/copoly(ethylene/butylene) type; styrene-methacrylate copolymers; modified clays; and mixtures thereof.

10. The cosmetic composition according to claim 9, wherein said liquid-fatty-phase gelling agent is chosen from the group consisting of MQ silicone resins; polymethylsesquioxanes and resins crosslinked via hydrosilylation; vinylic, acrylic or methacrylic copolymers; quaternium-18 bentonite; benzalkonium quaternium-18 bentonites; stearalkonium bentonites; and mixtures thereof.

11. The cosmetic composition according to claim 3, wherein the liquid-fatty-phase gelling agent comprises a non-crystalline, non-silicone fatty-phase gelling agent.

12. The cosmetic composition according to claim 1, wherein said liquid-fatty-phase gelling agent comprises an inorganic gelling agent.

13. The cosmetic composition according to claim 1, wherein said non-crystalline, non-silicone, liquid fatty-phase gelling agent is chosen from the group consisting of non-silicone polyamides; polyurethanes; (meth)acrylic and/or vinylic polymers; polymers or copolymers resulting from the polymerization or copolymerization of an ethylenic monomer and containing one or more ethylenic bonds; polymers or copolymers resulting from the polymerization or copolymerization of an ethylenic monomer, particularly vinylic, acrylic or methacrylic copolymers; triblock copolymers of polystyrene/polyisoprene or polystyrene/butadiene type, or of polystyrene/copoly(ethylene-propylene) type or of polystyrene/copoly(ethylene/butylene) type; styrene-methacrylate copolymers; and mixtures thereof.

14. The cosmetic composition according to claim 2, wherein said inorganic gelling agent is chosen from the group consisting of from modified clays, pyrogenic silica, and mixtures thereof.

15. The cosmetic composition according to claim 1 wherein said composition further comprises at least one colorant.

16. The cosmetic composition according to claim 2, wherein said colorant is chosen from organic pigments, mineral pigments, nacres and mixtures thereof.

17. The cosmetic composition according to claim 1, wherein said composition further comprises at least one silicone oil.

18. The cosmetic composition according to claim 4, wherein said silicone oil is chosen from:
linear or cyclic silicone oils, and
non-volatile polydimethylsiloxanes (PDMS), polydimethylsiloxanes containing pendant and/or terminal C₂-C₂₄ alkyl or alkoxy groups, phenyl silicones, and
mixtures thereof.

19. The cosmetic composition according to claim 4, wherein said silicone oil is chosen from:
linear or cyclic silicone oils having a viscosity≦8 centistokes (8×10⁻⁶ m²/s), octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and
non-volatile polydimethylsiloxanes (PDMS), polydimethylsiloxanes containing pendant and/or terminal C₂-C₂₄ alkyl or alkoxy groups phenyl trimethicones, phenyl dimethicones, phenyltrimethylsiloxydiphenylsiloxanes, diphenyl dimethicones, diphenylmethyldiphenyltrisiloxanes, and 2-phenylethyl trimethylsiloxysilicates, and
mixtures thereof.

20. The cosmetic composition according to claim 1, further comprising a hydrocarbon oil chosen from hydrogenated polyisobutene, stearyl heptanoate, isononyl isononanoate, dipentaerythritol tetrahydroxystearate/tetraisostearate, 2-octyldodecanol, isotridecyl isononanoate, diisostearyl malate, and mixtures thereof.

21. The cosmetic composition according to claim 1, further comprising a volatile oil chosen from:
hydrocarbon oils having from 8 to 16 carbon atoms,
linear or cyclic silicone oils,
fluorinated, and
mixtures thereof.

22. The cosmetic composition according to claim 21, wherein said volatile oil is present at less than 15% by weight relative to a total weight of the composition.

23. The cosmetic composition according to claim 1, further comprising at least one compound chosen from waxes, pasty fatty substances, and mixtures thereof.

24. The cosmetic composition according to claim 1, further comprising at least one filler.

25. The cosmetic composition according to claim 1, wherein the cosmetic composition comprises at least one of a skin care composition, a lip care composition and a makeup composition.

26. A method of preparing the anhydrous cosmetic composition of claim 1, comprising:
combining at least one liquid fatty phase, at least one gelling agent for said liquid fatty phase, and at least one silicone polymer of general formula (I):
R¹ₐR²_{b}R³_{c}SiO_{(4-a-b-c)/2} (I)
in which:
a is in a range of from about 1 to about 2.5,
b and c each are independently in a range of from about 0.001 to about 1.5,
R¹, which may be identical or different at each occurrence, is chosen from:
C₁ to C₃₀ alkyl radicals
C₁ to C₃₀ alkyl radicals, substituted by one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is chosen from C₁ to C₃₀ hydrocarbon radicals and radicals R⁵—(CO)—, in which R⁵ is chosen from C₁ to C₃₀ hydrocarbon radicals,
d is an integer in a range of from 0 to about 15, and
e and f are each integers that are independently in a range of from 0 to about 50,
and combinations thereof,
R² is a radical represented by the general formula (III):
-Q-O—X (III)
wherein:
Q is chosen from divalent C₂ to C₂₀ hydrocarbon radicals that can include at least one ether bond and/or at least one ester bond,
and R³ is an organosiloxane group of general formula (IV): 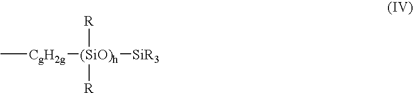
wherein:
radicals R are each radicals independently chosen from the group consisting of from C₁ to C₃₀ alkyl radicals, C₁ to C₃₀ alkyl radicals substituted by one or more fluorine atoms and aryl and aralkyl radicals,
g is an integer in a range of from 1 to about 5, and
h is an integer in a range from 0 to about 500, and
said gelling agent is a non-crystalline, non-silicone gelling agent.

27. A method of making up and/or caring for keratin materials comprising:
applying the cosmetic composition of claim 1 to one or more keratin materials chosen from the group consisting of skin, mucosae, nails, eyelashes and hair.
